(19) United States
(12) Patent Application Publication
(10) Pub. No.: US 2026/0338759 A1
(43) Pub. Date: Oct. 29, 2026

(54) Micro Magnetic Resonance Relaxometry (MRR) for Rapid and Non-Invasive Detection of IPSC Quality and Differentiation

(71) Applicants: Massachusetts Institute of Technology, Cambridge, MA (US); Nanyang Technological University, Singapore (SG); Singapore-MIT Alliance for Research & Technology Centre, Singapore (SG)

(72) Inventors: Jongyoon HAN, Bedford, MA (US); Sing Yian CHEW, Singapore (SG); Daniel ROXBY, Singapore (SG); Zu Yao TAN, Singapore (SG); Tan Dai NGUYEN, Singapore (SG)

(21) Appl. No.: 19/117,847

(22) PCT Filed: Oct. 3, 2023
(86) PCT No.: PCT/US23/75797

§ 371 Date: Apr. 2, 2025

Related U.S. Application Data
(60) Provisional application No. 63/412,640, filed on Oct. 3, 2022.

(51) Int. Cl. G01N 33/483; G01N 24/08; G01R 33/30; G01R 33/44

(52) U.S. Cl. CPC G01N 33/483; G01N 24/08; G01R 33/302; G01R 33/448

(57) ABSTRACT

Method and system wherein induced pluripotent stem cells quality and differentiation are evaluated using T2 magnetic resonance relaxometry.

### PRIORITY CLAIM

This application claims priority to U.S. Provisional Patent Application No. 63/412,253, filed Oct. 3, 2022, which is incorporated by reference in its entirety.

### FIELD OF THE INVENTION

The invention features systems and methods for stem cell analysis.

### BACKGROUND

A magnetic resonance relaxometry (MRR) device can measure the T₂ relaxation times of microlitre samples. See, for example, Peng, W. K., et al., 2020. Molecular phenotyping of oxidative stress in diabetes mellitus with point-of-care NMR system. NPJ Aging and Mechanisms of Disease, 2020, 6(1), pp.1-12; Fook Kong, T., et al. Enhancing malaria diagnosis through microfluidic cell enrichment and magnetic resonance relaxometry detection. Scientific Reports, 2015, 5(1), pp.1-12; Peng, W. K., et al. Development of miniaturized, portable magnetic resonance relaxometry system for point-of-care medical diagnosis. Review of Scientific Instruments, 2012 83(9), p.095115; and Thamarath, S. S., et al. 2022. Rapid and Live-cell Detection of Senescence in Mesenchymal Stem Cells by Micro Magnetic Resonance Relaxometry. bioRxiv doi.org/10.1101/2022.06.01.494362, each of which is incorporated by reference in its entirety. Briefly, the MRR device measures relaxation rates from spin echoes of the water content of cells using the same principles as Magnetic Resonance Imaging (MRI) or Nuclear Magnetic Resonance (NMR) with the distinct advantage of being able to use microlitre sized samples and minimal cell number requirements (~110-~180k cells per test), appropriate to the cell therapy context.

### SUMMARY

In general, magnetic resonance relaxometry (MRR) measurement can be used to evaluate induced pluripotent stem cells (iPSCs) and cells differentiated therefrom. For example, measurement of properties by MRR, particularly T₂, can be used to identify iPSCs with superior chondrogenesis, or cartilage regeneration capability. Cell types can be destinguished via their intracellular Fe³⁺ content. For example, OCT4 levels in SCPCs can be measured via the MRR system and was investigated. This measurement could provide an endpoint assessment of differentiation efficiencies in SCPC batch-to-batch production. In addition, T₂ measurements of iPSCs, as early as day 1 in the differentiation process, may be used to predict the differentiation efficiency outcome of iPSCs to SCPCs.

In one aspect, a method of evaluating induced pluripotent stem cells and cells differentiated therefrom can include loading a sample including a plurality of induced pluripotent stem cells and cells differentiated therefrom in a sensor, placing the sensor including the sample within or nearby a detection coil of a magnetic resonance relaxometry device, determining a T₂ value for the sample, and evaluating a quality of the induced pluripotent stem cells and cells differentiated therefrom from the T₂ value.

In another aspect, a system for evaluating induced pluripotent stem cells and cells differentiated therefrom can include a magnetic resonance relaxometry device configured to determining a T₂ value for a sample including a plurality of induced pluripotent stem cells and cells differentiated therefrom in a sensor and evaluating induced pluripotent stem cells by monitoring the T₂ value of the sample. In certain circumstances, the system can include a cell passaging device as a source for the plurality of induced pluripotent stem cells and cells differentiated therefrom. For example, a T₂ value of passaged induced pluripotent stem cells and cells differentiated therefrom can be measured to assess variation between the passaged induced pluripotent stem cells and cells differentiated therefrom from the induced pluripotent stem cells and cells differentiated therefrom.

In certain circumstances, evaluating can include comparing the T₂ value of the induced pluripotent stem cells and cells differentiated therefrom to a T₂ value of induced pluripotent stem cells and cells differentiated therefrom measured at an earlier time.

In certain circumstances, the T₂ value can correlate to a percentage of differentiated progenitor cells.

In certain circumstances, the T₂ value can correlate to a percentage of undifferentiated, residual iPSCs in a differentiated cell population.

In certain circumstances, the T₂ value can correlate to an ability of iPSCs to differentiate into progenitor cells or fully differentiated cells.

In certain circumstances, the T₂ value can correlate to an EdU colony forming assay.

In certain circumstances, evaluating a quality of the induced pluripotent stem cells and cells differentiated therefrom, from the T₂ value occurs on day 1 of a differentiation process. In certain circumstances, evaluating a quality of the induced pluripotent stem cells and cells differentiated therefrom, from the T₂ value occurs on day 4 of a differentiation process. In certain circumstances, evaluating a quality of the induced pluripotent stem cells and cells differentiated therefrom, from the T₂ value occurs on day 10 of a differentiation process. In other circumstances, evaluating can be on a combination of day 1 and day 10, day 1 and day 4, day 4 and day 10, or day 1, day 4 and day 10.

In certain circumstances, the induced pluripotent stem cells and cells differentiated therefrom are suitable for spinal cord injury cell therapy applications.

In certain circumstances, the method can include passaging induced pluripotent stem cells and cells differentiated therefrom, measuring a T₂ value of the passaged induced pluripotent stem cells and cells differentiated therefrom to assess variation between the passaged induced pluripotent stem cells and cells differentiated therefrom from the induced pluripotent stem cells and cells differentiated therefrom.

In certain circumstances, the sample can be a pellet including induced pluripotent stem cells and cells differentiated therefrom in the sensor.

In certain circumstances, the sample can include a cluster of induced pluripotent stem cells and cells differentiated therefrom.

In certain circumstances, the cluster can have a size of at least 50 microns.

In certain circumstances, the T₂ value can correlate to a doubling time for the induced pluripotent stem cells and cells differentiated therefrom.

In certain circumstances, the T₂ value can correlate to a differentiated stem cell.

In certain circumstances, the T₂ value can correlate to OCT4 expression.

In certain circumstances, the sensor can be a tube or chamber.

In certain circumstances, the magnetic resonance relaxometry device can include a radio frequency probe.

In certain circumstances, determining the T₂ value can include supplying a train of pulses over a period of less than one minute.

In certain circumstances, determining the T₂ value can include obtaining and averaging 2 to 70 scans, 4 to 60 scans, or 6 to 40 scans. For example, determining the T₂ value can include obtaining and averaging 10 to 30 scans.

In certain circumstances, a detection region of the magnetic resonance relaxometry device can include a volume of less than about 1 μL of the sample. For example, the volume can be less than 0.1 μL, less than about 0.01 μL, less than about 0.001 μL, or less than about 0.0001 μL, In certain circumstances, the volume can be about 1 pL to 10 pL.

In certain circumstances, the liquid sample can be free of paramagnetic or ferromagnetic materials, including paramagnetic or ferromagnetic metal ions or compounds thereof.

In certain circumstances, the cell passaging device can be a source for the plurality of induced pluripotent stem cells and cells differentiated therefrom. For example, the cell production device can include a cell culture system.

In certain circumstances, the liquid sample can be contained in a microcapillary.

Other aspects, embodiments, and features will be apparent from the following description, the drawings, and the claims.

### BRIEF DESCRIPTION OF DRAWINGS

FIG. 1 depicts a schematic of a Magnetic Resonance Relaxometry (MRR) device.

FIG. 2 depicts a schematic of system including a Magnetic Resonance Relaxometry (MRR) device and a cell production device as a source of stem cells.

FIGS. 3A-3B show a CPMG (Carr-Purcell-Meiboom-Gill) pulse sequence for measuring the T₂ values. FIG. 3C is a schematic depicting spin-spin relaxation time of iPSCs changing as they differentiate into SCPCs.

FIG. 4 is a schematic depicting sample preparation process for the iPSCs and spinal cord progenitor cells (SCPCs) being tested in the MRR instrument in suspended form.

FIG. 5 is a schematic depicting sample preparation process for the iPSCs and SCPCs being tested in the MRR instrument in pelleted form.

FIG. 6 is a graph depicting T₂ relaxation times over several passages under normal culturing conditions. Data accumulated over 6 months of iPSCs generated herein.

FIG. 7 is a graph depicting T₂ relaxation times over several passages under normal controlled single cell culturing conditions. One culture was split into two separate cultures. Each culture was grown between 8 different wells and seeded at a density of 500k cells per well. After three days of growth, a portion of the cells were taken for magnetic testing using method 1 and cell counting, whilst the rest were re-seeded for the next passage.

FIG. 8 is a graph depicting T₂ relaxation times over several passages under normal controlled culturing conditions.

FIG. 9 is a graph depicting T₂ relaxation times measured with method 2, for three cultures plotted against population doubling times.

FIG. 10 is a graph depicting T₂ relaxation times for iPSCs continuously passaged vs frozen and thawed. In this experiment, magnetic measurements on the left were on cells that were continuously passaged from P38 through to P40. Measurements on right were from cells that had undergone cryopreservation. This process involves the storing of cells in media, ROCKi and DMSO at −80 degrees C to acclimatise them, before being transferred into liquid nitrogen for long term storage. The cells are then thawed and re-plated to be grown as per normal protocols.

FIG. 11 is a graph depicting correlation between percentage of large clumps seeded in a culture and the population doubling time.

FIG. 12 is a graph depicting day 10 values of OCT4 vs MRR T₂ Relaxation Time. T₂ relaxation time values increase as OCT4 value decreases.

FIG. 13 is a graph depicting OCT4 residual expression on day 10 vs T₂ relaxation times on day 1. Predicting day 10 OCT4 values based on MRR measurements on day 1.

FIGS. 14A-14E depict changes in expression of OCT4 and SOX1, as well as T₂ measurements as iPSCs differentiated to SCPCs. FIG. 14A shows immunofluorescent staining to illustrate changes in Oct4 and Sox1 phenotypes during the differentiation of iPSCs into SCPCs across 3 different timepoints (Day 1, day 4 and day 10). DAPI (blue, nuclear), SOX1 (green, neural progenitor marker) and OCT4 (red, pluripotent marker (Scale bar: 50 μm). FIG. 14B shows OCT4⁺ cells decrease as iPSCs differentiate to day 10 SCPCs. FIG. 14C shows increase in T₂ as iPSCs differentiated to day 10 SCPCs. FIG. 14D shows an increase in T₂ with decreasing level of OCT4⁺ cells across 10-day differentiation period. FIG. 14E show CLEC23 iPSCs and SCPCs stained with Fe³⁺ reversible fluorescent sensor shows that the fluorescence intensity of the stained cells via FACS analysis corresponds to their T₂ (n=2). At least 10 ROIs (more than 60,000 cells) were quantified for each experimental repeat (n=4). Statistical significance was determined by a non-parametric Kruskal-Wallis test followed by a Dunn post-hoc test.

FIGS. 15A-15C depict the presence of artificially spiked iPSCs in the SCPC population decreases measured T₂ values in a dose-dependent manner. FIG. 15A shows artificial spiking of iPSCs in SCPCs at 1%, 5% and 10% concentration (n=4). FIG. 15B shows immunofluorescent staining of SCPCs with spiked iPSCs subjected to colony culture assay. Staining includes DAPI (blue, nuclear) and OCT4 (red, pluripotent marker). An increasing number of iPSC colonies and colony sizes were observed with increasing spiked iPSC concentrations (n=2). FIG. 15C shows quantification of iPSC colony sizes with frequency distribution plot of colony area size (n=2). At least 100 ROIs (more than 100,000 cells) were quantified for each experimental repeat (n=3). Statistical significance was determined by a Mann-Whitney u-test.

FIGS. 16A-16C depict correlation of day 10 T₂ to OCT4⁺ levels and day 1 T₂ to day 10 T₂ and day 10 OCT4⁺ levels. Pearson's correlation coefficient is a statistical measure of the strength of a linear relationship between paired data, and was employed to benchmark the T₂ relaxation time measurements with OCT4⁺ cells quantified by immunofluorescence staining. FIG. 16A shows correlation of day 10 SCPC T₂ with the percentage of day 10 OCT4⁺ cells quantified by immunofluorescent staining. FIG. 16B shows correlation of day 1 undifferentiated iPSC T₂ to day 10 SCPC T₂. FIG. 16C shows correlation of day 1 undifferentiated iPSC T₂ with the percentage of day 10 OCT4⁺ cells quantified by immunofluorescent staining. At least 10 ROIs (more than 60,000 cells) were quantified for each experimental repeat (n=4).

FIGS. 17A-17G depict decreased concentration of CHIR results in poor differentiation efficiency of iPSCs to SCPCs with corresponding decrease in T₂. FIGS. 17A-17C show FACS analysis of SOX1⁺, Nestin⁺ and OCT4⁺ cells with decreasing CHIR concentration (n=3). FIG. 17A shows a decrease in SOX1⁺ cells with decreasing CHIR concentration. FIG. 17B shows a decrease in Nestin⁺ cells with decreasing CHIR concentration. FIG. 17C shows an increase in OCT4⁺ cells with decreasing CHIR concentration. FIG. 17D shows T₂ decreases with decreasing CHIR concentration (n=3). FIG. 17E shows T₂ decreases with decreasing SCPC SOX1⁺ levels (n=3). FIG. 17F shows T₂ decreases with decreasing SCPC Nestin⁺ levels (n=3). FIG. 17G shows T₂ decreases with increasing SCPC OCT4⁺ levels (n=3). Statistical significance was determined by an independent paired 2-tailed t-test.

FIGS. 18A-18C depict comparison of CLEC23 iPSCs and BJ-iPSCs. FIG. 18A shows CLEC23 iPSCs and BJ iPSCs exhibit similar ranges of T₂ relaxation time and OCT4 levels. FIG. 18B shows Day 10 BJ-SCPCs have a higher percentage of SOX1⁺ cells and T₂ relaxation time as compared to day 10 CLEC23 SCPCs (n=3). FIG. 18C show Day 10 BJ-SCPCs have a lower percentage of OCT4⁺ cells and higher T₂ relaxation time compared to day 10 CLEC23 SCPCs (n=3). At least 25 ROIs (more than 60,000 cells) were quantified for each experimental repeat (n=3).

### DETAILED DESCRIPTION

As described herein, and building on the description of the device described above, a method of evaluating induced pluripotent stem cells and cells differentiated therefrom can include loading a sample including a plurality of induced pluripotent stem cells and cells differentiated therefrom in a sensor, placing the sensor including the sample within or nearby a detection coil of a magnetic resonance relaxometry device, determining a T₂ value for the sample, and evaluating a quality of the induced pluripotent stem cells and cells differentiated therefrom from the T₂ value. The quality of the iPSCs can be measure of a critical quality attribute of the iSPCs. For example, the population doubling time, the variability of cells from passage to passage, and the degree of differentiation observed in the iPSCs can each be a quality that is important in evaluating the health, nature, or status of the iSPCs. Decreases in T₂ can indicate a weak or defective plurality of iSPCs.

For example, the advent of induced pluripotent stem cells (iPSCs) has provided a promising solution to replacing damaged neurons and glial cells, especially in the case of spinal cord injuries (SCI). Despite its merits, iPSC and its differentiation into neural progenitors have been a variable process, prompting the need to reliably assess the degree of differentiation achieved in any given batch of cells and to validate the quality and safety of the differentiated cells. In this study, human iPSCs were differentiated into spinal cord progenitor cells (SCPCs). During the 10-day differentiation process, intracellular iron content (i.e., Fe³⁺) were measured at different timepoints using Magnetic Resonance Relaxometry (MRR) in a rapid and label-free manner via its T₂ relaxation time. It was found that SCPC batches containing higher levels of pluripotent markers (i.e., OCT4) have lower T₂ (p<0.005) than SCPC batches with lower levels of these markers. Furthermore, by negatively affecting the differentiation of iPSCs to SCPCs, groups with lower levels of neural progenitor (i.e., SOX1) and stem (i.e., Nestin) cell have lower T₂ (p<0.005) than normally differentiated SCPCs. Our technology provides a rapid label-free method to determine critical quality attributes in iPSC-derived progenies, ideally suited as a quality control tool in cell therapy manufacturing.

In another aspect, a system for evaluating induced pluripotent stem cells and cells differentiated therefrom can include a magnetic resonance relaxometry device configured to determining a T₂ value for a sample including a plurality of induced pluripotent stem cells and cells differentiated therefrom in a sensor and evaluating induced pluripotent stem cells and cells differentiated therefrom by monitoring the T₂ value of the sample. In certain circumstances, the system can include a cell passaging device as a source for the plurality of induced pluripotent stem cells and cells differentiated therefrom. For example, a T₂ value of passaged induced pluripotent stem cells can be measured to assess variation between the passaged induced pluripotent stem cells and cells differentiated therefrom from the induced pluripotent stem cells and cells differentiated therefrom.

In certain circumstances, evaluating can include comparing the T₂ value of the induced pluripotent stem cells and cells differentiated therefrom to a T₂ value of induced pluripotent stem cells and cells differentiated therefrom measured at an earlier time.

In certain circumstances, the method can include passaging induced pluripotent stem cells and cells differentiated therefrom, measuring a T₂ value of the passaged induced pluripotent stem cells and cells differentiated therefrom to assess variation between the passaged induced pluripotent stem cells and cells differentiated therefrom from the induced pluripotent stem cells and cells differentiated therefrom.

In certain circumstances, the sample can be a pellet including induced pluripotent stem cells and cells differentiated therefrom in the sensor. The pellet can be positioned in the sensor at a location to improve detected signal for the T₂ measurement.

In certain circumstances, the sample can include a cluster of induced pluripotent stem cells and cells differentiated therefrom. For example, the sample can include one or more cluster. The cluster can have a size of at least 50 microns, at least 55 microns, at least 60 microns, at least 65 microns, at least 70 microns, at least 75 microns, at least 80 microns, at least 85 microns, or at least 90 microns. In certain circumstances, the cluster can have a maximum size of 250 microns.

In certain circumstances, the T₂ value can correlate to a doubling time for the induced pluripotent stem cells and cells differentiated therefrom. The doubling time can be an indicator of the health of the cells. For example, a decrease in doubling time over a measured period of time can indicate that the iSPCs may be differentiating and may no longer be suitable for a particular purpose.

The systems and methods described herein relate to induced pluripotent stem cells and to cells differentiated therefrom. For example, the induced pluripotent stem cells can be differentiated stem cells.

In certain circumstances, the T₂ value can correlate to a percentage of differentiated progenitor cells.

In certain circumstances, the T₂ value can correlate to a percentage of undifferentiated, residual iPSCs in a differentiated cell population.

In certain circumstances, the T₂ value can correlate to an ability of iPSCs to differentiate into progenitor cells or fully differentiated cells.

In certain circumstances, the T₂ value can correlate to an EdU colony forming assay.

In certain circumstances, the T₂ value can correlate to a differentiated stem cell. For example, the T₂ value or progression of T₂ value of a sample can lead to establishing a quality control threshold that can improve the efficiency of using the cells.

In certain circumstances, evaluating a quality of the induced pluripotent stem cells and cells differentiated therefrom, from the T₂ value occurs on day 1 of a differentiation process. In certain circumstances, evaluating a quality of the induced pluripotent stem cells and cells differentiated therefrom, from the T₂ value occurs on day 4 of a differentiation process. In certain circumstances, evaluating a quality of the induced pluripotent stem cells and cells differentiated therefrom, from the T₂ value occurs on day 10 of a differentiation process. In other circumstances, evaluating can be on a combination of day 1 and day 10, day 1 and day 4, day 4 and day 10, or day 1, day 4 and day 10.

In certain circumstances, the induced pluripotent stem cells and cells differentiated therefrom are suitable for spinal cord injury cell therapy applications.

In certain circumstances, the T₂ value can correlate to protein expression from the iSPCs or from the SCPCs. For example, OCT4 expression can indicate healthy behaviour of the cells.

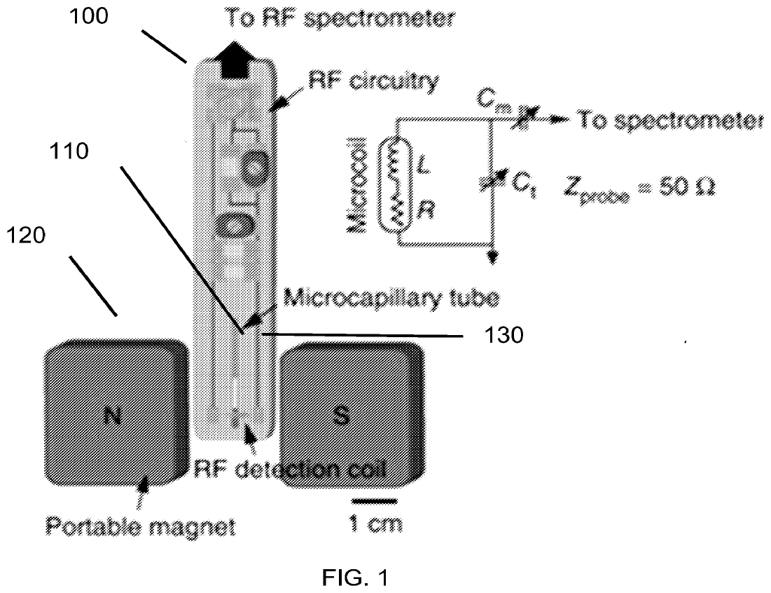

A device for performing magnetic resonance relaxometry is described, for example, in U.S. Pat. No. 10,429,467, which is incorporated by reference in its entirety. Referring to FIG. 1, a device can include an MRR system. FIG. 1 is a schematic of a Magnetic Resonance Relaxometry (MRR) system 100 in accordance with one aspect of this disclosure. The system 100 can include a Field-Programmable Gate Array-based (FPGA-based) radio frequency (rf) spectrometer to control the MRR system 100, a first direct digital synthesis module for generation of radio frequency pulses, a transmitter (TRANS) for transmission of the generated radio frequency pulses to a radio frequency (rf) probe and detection coil 110, a receiver (RCVR) for receiving resonance information from the radio frequency probe, a first power amplifier (PA), a pre-amplifier (p-amp), a duplexer (Dup) for transmitting a high power excitation pulse to the rf probe in the transmission mode and for isolating the high power excitation pulse from the receiver during receiving mode, and a magnet system 120. A sample 130 can be placed in a sensor, surch as a tube or chamber, for example, a microcapillary tube, that can be positioned in an RF detection coil. In many embodiments, the FPGA-based rf spectrometer can include a pulse programmer (PPG) adapted to control the FPGA-based rf spectrometer and a second direct digital synthesis (DDS). The second DDS can generate a fixed intermediate frequency (IF). The first DDS can be configured to generate a variable desired frequency. In accordance with one aspect of this disclosure, the FPGA-based spectrometer may use the design set forth in Takeda K. (2007), “A highly integrated FPGA-based nuclear magnetic resonance spectrometer,” Rev Sci Instrum 78(3):033103; and/or in Takeda K. (2008)“OPENCORE NMR: open-source core modules for implementing an integrated FPGA-based NMR spectrometer,” Journal of Magnetic Resonance 192(2):218-229, the teachings of which two references are incorporated by reference in their entirety.

In order to facilitate processing of information to and from the MRR system 100, the FPGA-based rf spectrometer is couplable to at least one external electronic device which may, for example, include a personal computer, mobile phone and/or a portable electronic tablet. Coupling between the MRR system 100 and the at least one external electronic device may be by way of at least one of USB, HDMI and/or wireless connection means such as Wi-Fi and/or Bluetooth.

In conventional NMR systems, the major cost of instrumentation lies on the superconducting magnet (or permanent magnet) and rf-spectrometer. In accordance with one aspect of this disclosure, the whole system may cost less than $2500; in which the majority of the cost lies on the FPGA chip ($1000 each), external GHz-clock ($250 each), DDS (Analog-Device; AD9858, $400 each), 1-Watt power amplifier ($100), pre-amplifier ($50), RCVR (AD8343, $4 each), TRANS (AD834, $20 each, and AD8343) and USB (FT2232D, $10 each). Indicated in the parentheses is the cost of the main electronic component used. Others periphery components such as pin connectors (e.g., SMA), capacitors, rf-switches, rf-transformers and rf-filters cost less than $10 each.

The MRR system 100 may be adaptable to operate in various modes to detect NMR-active nuclei such as proton, fluorine, phosphorus and carbon. The magnetic field used in each mode in which the MRR system 100 operates depends on which nuclei are to be detected. Depending on the mode of operation, the MRR system 100 can operate at a magnetic field of between approximately 0.1 and 3 Tesla (T) which can correspond to between approximately 1 and 150 MHz. For instance, when the MRR system 100 is operating in a proton NMR mode, the magnetic field is approximately 0.76 T which corresponds to approximately 31.9 MHz for proton NMR frequency.

The MRR system 100 can be controlled by the FPGA-based rf spectrometer which comprises the pulse programmer and the second DDS. As compared to CMOS technology, FPGA provides the advantages of re-programmability. The FPGA-based rf spectrometer may, for example, be programmable using tools and software provided by vendors such as Altera Corporation of San Jose, Calif., U.S.A. and Xilinx, Inc. of San Jose, Calif., U.S.A. In an exemplary embodiment, the FPGA chip can include the EP3C80F780C8N, Cyclone III (Altera) embedded on a breadboard (ACM-202-80C8, HumanData, Japan). This chip has 81000 logic elements and is capable of producing 3 independent if-outputs, when fully utilized.

The pulse programmer can generate high power excitation rf pulses. The generated rf pulses then pass through the first power amplifier to produce optimized rf-power for a duration of approximately between 1 and 1000 microseconds to excite all the nuclei effectively. The high power rf pulses are transmitted to the rf probe and will be discussed further herein.

In an exemplary operation, power used for liquid state and solid-state NMR is approximately between 0.1 W and 10 W and approximately between 100 W and 1000 W, respectively. A “strong” power amplifier is often indispensable in MRR systems and such “strong” power amplifiers are often bulky, and require high power consumption, thereby posing serious limitation for field work. For example, a novel and lightweight 1-Watt power amplifier can be constructed on a 4 cm by 4 cm printed circuit board. A solenoid type microcoil (inner diameter 700 to 1000 μm, for example, 750, 800, 850, 900 or 950 μm) can be further employed to generate a strong oscillating magnetic field, B₁, and picks up a signal from the free induction decay (FID) or spin-echo. By employing the duplexer, the high power excitation if pulses that are to be transmitted to the rf-probe in the transmission mode can be isolated from the receiver or detection coil 110 during the receiving mode. The FID/spin-echo is then amplified by a pre-amplifier (AMP-75+, Mini Circuits, USA) with a gain of 20 dB and noise figure of 2.83, and finally filtered by appropriate low pass filter before going into the receiver circuit. FID is the observable NMR signal generated by non-equilibrium nuclear spin magnetization precessing about the static magnetic field (conventionally along z-axis). This non-equilibrium magnetisation can be induced, by applying a pulse of resonant radio-frequency close to the Larmor frequency of the nuclear spins. Spin-echo is the refocusing pulse after a single 90-degree inversion followed by inverting them by an 180-degree pulse at resonant.

The magnet system 120 may be portable and light weight (for example, about 60 g) and adaptable to produce a high static field. The magnet system 120 may comprise at least one magnet disposed adjacent to the rf probe. Alternative embodiments include having at least two magnets disposed adjacent to the rf probe. The rf probe can be disposed between the at least two magnets. The magnet system 120 can comprise a permanent magnet and/or an electromagnet. Permanent magnets used in the magnet system 120 may, for example, include Neodymium based magnets.

A detection region of the magnetic resonance relaxometry device can include a volume of less than about 1 μl of the sample for detection. For example, the sample can be provided in sensor, such as a capillary tube or microcapillary tube or a chamber. The sample can be sealed from the ambient environment, reducing exposure to oxygen and other materials that could negatively impact the ability of the magnetic resonance relaxometry device to detect cells. The sample can include a buffer solution that is free of any paramagnetic iron compounds, which can be important in order to give an accurate result.

The current μMRR set up is only suitable for measurement of cell suspension, for example, in a sample. A μMRR set up can be designed to adherent cells on cell culture plates. The devices, systems and methods described herein can be used to develop and improve cell therapies by facilitating removal of cells from cell populations through simplifying the identification process for cells and using that approach in conjunction with cell manipulation technologies.

A number of approaches can be taken to improve the accuracy of the T₂ value. For example, determining the T₂ value can include obtaining and averaging a plurality of scans. Up to 100 (or more) scans can be averaged. The number of scans can be less than 80, less than 70, or less than 60. More typically, 10 to 50 scans can be averaged. As a minimum, under certain circumstances, 2 scans, 4 scans, 5 scans, 8 scans, 10 scans, 12 scans, 14 scans, 16 scans, 18 scans, 20 scans, 22 scans, 24 scans, 26 scans, 28 scans, or 30 scans can be averaged.

The systems and methods described herein can be used to develop an assay. The assay could be used as a quality control assay during iPSC production. Alternatively, the assay can be done in the middle of the cell production, monitoring the progress of the culture and identify any deviation from the range of acceptable values, to identify the bad batches early. The assay can be used in conjunction with a cell production device can be a source for the plurality of iSPCs. For example, the cell passaging device can include a cell culture system.

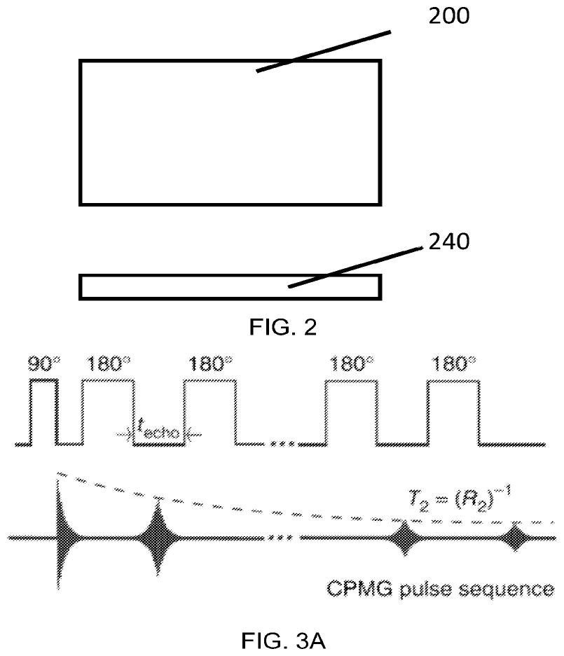

Referring to FIG. 2, a system described herein can include magnetic resonance relaxometry device 200 and cell passaging device 240. For example, a system for identifying chondrogenic potential of mesenchymal stem cells can include a magnetic resonance relaxometry device configured to determine a T₂ value for the liquid sample, and evaluating induced pluripotent stem cells by monitoring the T₂ value of the sample.

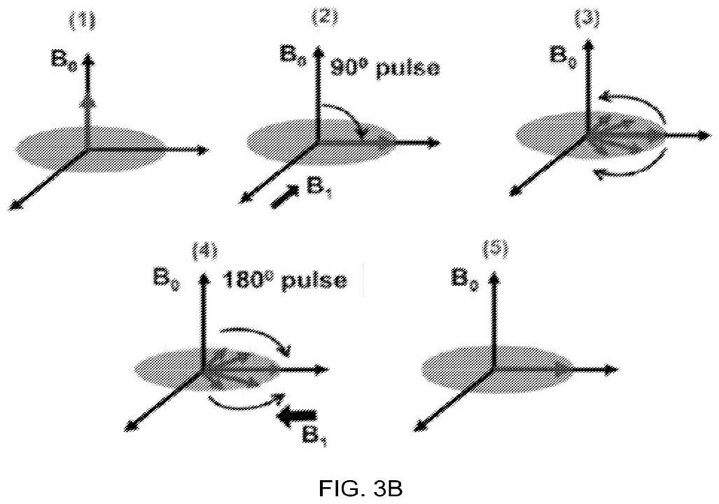

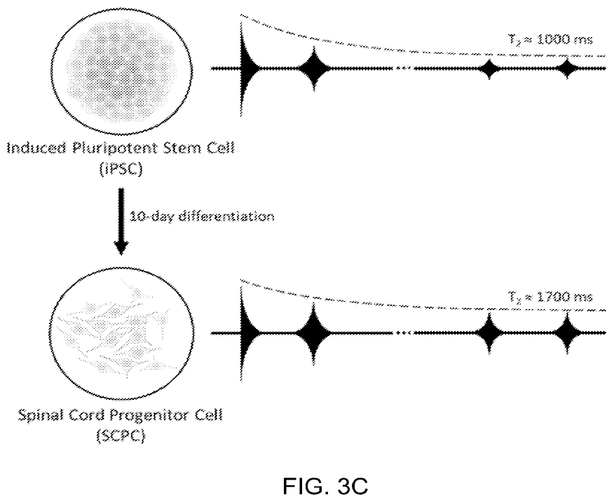

Referring to FIGS. 3A and 3B, the CPMG (Carr-Purcell-Meiboom-Gill) pulse sequence for measuring the T₂ values can work efficiently in inhomogeneous magnetic fields produced by permanent magnet of MRR. A train of radiofrequency pulses are applied to the proton nuclei at the resonance frequency of 21.65 MHz with the inter echo time interval t echo and it is repeated for thousands of echoes until relaxes over time. This decayed height of echoes over time is called transverse relaxation time T₂.

FIG. 1 shows non-invasive and rapid Magnetic resonance relaxometry (MRR) measurement to evaluate induced pluripotent stem cells. FIG. 1 is a schematic showing a MRR system consists of a permanent magnet that provides a strong magnetic field (B₀). A home-built radiofrequency (RF) detection probe is connected to an RF spectrometer. The iPSC sample in the microcapillary tube is placed in the RF detection coil for T₂ measurements. FIG. 1B shows a microcapillary tube contains the 4 μl of cell sample in the 4 mm detection range of the RF detection coil. The typical cell number required is 60,000 cells within the detection volume. The bottom grey color is the crystoseal to seal the microcapillary tube.

FIG. 2 depicts a schematic of system including a Magnetic Resonance Relaxometry (MRR) device and a cell passaging device as a source of plurality of induced pluripotent stem cells.

FIGS. 3A and 3B show a CPMG (Carr-Purcell-Meiboom-Gill) pulse sequence for measuring the T₂ values, which works efficiently in inhomogeneous magnetic fields produced by the permanent magnet of μMRR. A train of radiofrequency pulses is applied to the proton nuclei at the resonance frequency of 21.65 MHz with the inter echo time interval techo. It is repeated for thousands of echoes until an equilibrium is reached. This decaying peak height of successive echoes over time is called transverse relaxation time T₂. FIG. 3C shows a schematic depicting spin-spin relaxation time of iPSCs changing as they differentiate into SCPCs.

The development of induced pluripotent stem cells (iPSC) by Yamanaka and his colleagues has ushered in a revolutionary age for stem cell therapy. See, K. Takahashi and S. Yamanaka, ‘Induction of Pluripotent Stem Cells from Mouse Embryonic and Adult Fibroblast Cultures by Defined Factors’, Cell, vol. 126, no. 4, pp. 663-676, August 2006, which is incorporated by reference in its entirety. iPSCs have similar abilities to embryonic stem cells (ESC) and can multiply indefinitely and differentiate into any somatic cell line under the right conditions. See, S. Yamanaka, ‘Pluripotent Stem Cell-Based Cell Therapy—Promise and Challenges’, Cell Stem Cell, vol. 27, no. 4, pp. 523-531, October 2020, which is incorporated by reference in its entirety. Furthermore, iPSCs serve as a desirable alternative to ESCs as the latter originate from embryos, and their use can be controversial in various settings and are limited in supply. Therapeutic applications of cell and tissue regeneration, enabled by iPSCs, that are currently ongoing in clinical trials include Parkinson's disease, macular degeneration, heart failure, spinal cord injury, platelet transfusion, cartilage defects, and cancer immunotherapy. See, for example, S. Yamanaka, ‘Pluripotent Stem Cell-Based Cell Therapy—Promise and Challenges’, Cell Stem Cell, vol. 27, no. 4, pp. 523-531, October 2020, J. Takahashi, ‘iPS cell-based therapy for Parkinson's disease: A Kyoto trial’, Regen. Ther., vol. 13, pp. 18-22, March 2020, I. W. Caras, L. R. Collins, and A. A. Creasey, ‘A Stem Cell Journey in Ophthalmology: From the Bench to the Clinic’, Stem Cells Transl. Med., vol. 10, no. 12, pp. 1581-1587, December 2021, S. Miyagawa et al., ‘Case report: Transplantation of human induced pluripotent stem cell-derived cardiomyocyte patches for ischemic cardiomyopathy’, Front. Cardiovasc. Med., vol. 9, p. 950829, August 2022, K. Sugai et al., ‘First-in-human clinical trial of transplantation of iPSC-derived NS/PCs in subacute complete spinal cord injury: Study protocol’, Regen. Ther., vol. 18, pp. 321-333, December 2021, N. Sugimoto et al., ‘iPLAT1: the first-in-human clinical trial of iPSC-derived platelets as a phase 1 autologous transfusion study’, Blood, vol. 140, no. 22, pp. 2398-2402, December 2022, K. Abe et al., ‘Engraftment of allogeneic iPS cell-derived cartilage organoid in a primate model of articular cartilage defect’, Nat. Commun., vol. 14, no. 1, Art. no. 1, February 2023, and D. Hong et al., ‘380 Preliminary results of an ongoing phase I trial of FT500, a first-in-class, off-the-shelf, induced pluripotent stem cell (iPSC) derived natural killer (NK) cell therapy in advanced solid tumors’, J. Immunother. Cancer, vol. 8, no. Suppl 3, November 2020, each of which is incorporated by reference in its entirety. iPSC is especially important for spinal cord injuries (SCI), where current treatments for SCI are severely lacking in reversing damage to the spinal cord, and have only primarily focused on surgery for spinal realignment and subsequent rehabilitation. See, for example, N. Nagoshi, O. Tsuji, M. Nakamura, and H. Okano, ‘Cell therapy for spinal cord injury using induced pluripotent stem cells’, Regen. Ther., vol. 11, pp. 75-80, June 2019, which is incorporated by reference in its entirety. A promising alternative for SCI treatments is the use of iPSC cell therapy which generates cells necessary to replace damaged neurons and glial cells, ensuring effective spinal cord regeneration. See, for example, M. Nakamura and H. Okano, ‘Cell transplantation therapies for spinal cord injury focusing on induced pluripotent stem cells’, Cell Res., vol. 23, no. 1, Art. no. 1, January 2013, which is incorporated by reference in its entirety. Several research groups have successfully demonstrated the transplantation of iPSC-derived neural progenitor cells into rodent and monkey animal models of SCI, resulting in the restoration of their motor function. See, for example, M. Nakamura and H. Okano, ‘Cell transplantation therapies for spinal cord injury focusing on induced pluripotent stem cells’, Cell Res., vol. 23, no. 1, Art. no. 1, January 2013, S. Nori et al., ‘Grafted human-induced pluripotent stem-cell-derived neurospheres promote motor functional recovery after spinal cord injury in mice’, Proc. Natl. Acad. Sci., vol. 108, no. 40, pp. 16825-16830, October 2011, and A. Yasuda et al., ‘Significance of Remyelination by Neural Stem/Progenitor Cells Transplanted into the Injured Spinal Cord’, STEM CELLS, vol. 29, no. 12, pp. 1983-1994, 2011 each of which is incorporated by reference in its entirety.

For SCI-related iPSC cell therapy, neural progenitor and stem cells (NPC/NSC) such as spinal cord progenitor cells (SCPCs) are differentiated from various established iPSC cell lines on a Matrigel-coated tissue culture plastic for a certain time period that differs from protocol to protocol, which is incorporated by reference in its entirety. See, for example, S. H. Tay, Winanto, Z. J. Khong, Y. H. Koh, and S. Y. Ng, ‘Generation of Cortical, Dopaminergic, Motor, and Sensory Neurons from Human Pluripotent Stem Cells’, in Methods in Molecular Biology. New York, NY: Springer US, 2021, which is incorporated by reference in its entirety. This process differentiates the iPSCs into NPCs, which are then directly implanted into the injury site of the spinal cord or as part of a tissue scaffolding. See, for example, J. E. Shin et al., ‘Brain and spinal cord injury repair by implantation of human neural progenitor cells seeded onto polymer scaffolds’, Exp. Mol. Med., vol. 50, no. 4, p. 39, April 2018, which is incorporated by reference in its entirety. Though the differentiation process of iPSCs into SCPCs is relatively efficient (typically >80% differentiation efficiency at day 10), the risk of tumorigenicity post-transplantation remains a considerable concern. See, for example, Q. Qu et al., ‘High-efficiency motor neuron differentiation from human pluripotent stem cells and the function of Islet-1’, Nat. Commun., vol. 5, no. 1, Art. no. 1, March 2014, Z.-W. Du et al., ‘Generation and expansion of highly pure motor neuron progenitors from human pluripotent stem cells’, Nat. Commun., vol. 6, no. 1, Art. no. 1, March 2015, N. Balafkan et al., ‘A method for differentiating human induced pluripotent stem cells toward functional cardiomyocytes in 96-well microplates’, Sci. Rep., vol. 10, no. 1, Art. no. 1, October 2020, U. Ben-David and N. Benvenisty, ‘The tumorigenicity of human embryonic and induced pluripotent stem cells’, Nat. Rev. Cancer, vol. 11, no. 4, pp. 268-277, April 2011, and S. Nori et al., ‘Long-Term Safety Issues of iPSC-Based Cell Therapy in a Spinal Cord Injury Model: Oncogenic Transformation with Epithelial-Mesenchymal Transition’, Stem Cell Rep., vol. 4, no. 3, pp. 360-373, March 2015, each of which is incorporated by reference in its entirety. Moreover, even though the use of several iPSC lines has been validated and established to be safe, the variations in differentiation efficiency or tumor-like overgrowth are still a recurring problem when iPSCs are differentiated into neural progenitors and subsequently transplanted into injured spinal cords. See, for example, S. Nori et al., ‘Long-Term Safety Issues of iPSC-Based Cell Therapy in a Spinal Cord Injury Model: Oncogenic Transformation with Epithelial-Mesenchymal Transition’, Stem Cell Rep., vol. 4, no. 3, pp. 360-373, March 2015, which is incorporated by reference in its entirety. A small number of residual undifferentiated iPSC-like cells pose a significant safety risk due to their potential neoplasticity in the implanted tissue. See, for example, J. Carlos and A. L., ‘Safety Assessment of Reprogrammed Cells Prior to Clinical Applications: Potential Approaches to Eliminate Teratoma Formation’, in Pluripotent Stem Cells, D. Bhartiya, Ed., InTech, 2013, which is incorporated by reference in its entirety.

Previously, micro magnetic resonance relaxometry (MRR) measurements has been used as a non-labeled assay for malaria infection, oxidated stress in the blood, and very recently to detect senescence in MSCs. See, for example, W. K. Peng et al., ‘Micromagnetic resonance relaxometry for rapid label-free malaria diagnosis’, Nat. Med., vol. 20, no. 9, pp. 1069-1073, September 2014, W. K. Peng, L. Chen, B. O. Boehm, J. Han, and T. P. Loh, ‘Molecular phenotyping of oxidative stress in diabetes mellitus with point-of-care NMR system’, Npj Aging Mech. Dis., vol. 6, no. 1, Art. no. 1, October 2020, and S. S. Thamarath et al., ‘Rapid and Live-cell Detection of Senescence in Mesenchymal Stem Cells by Micro Magnetic Resonance Relaxometry’, Biophysics, preprint, June 2022, each of which is incorporated by reference in its entirety. The spin-spin relaxation time (T₂) measurement is sensitive to the paramagnetic content of the sample, and in the context of cell biology, it is correlated with the intracellular content of iron (Fe³⁺). See, for example, M. S. Petronek et al., ‘Quantum chemical insight into the effects of the local electron environment on T2*-based MRI’, Sci. Rep., vol. 11, no. 1, p. 20817, October 2021, which is incorporated by reference in its entirety. In this study, a novel use of MRR for rapid endpoint analysis of iPSC and SCPC phenotypes closely related to their safety and quality parameters is reported. Thus, MRR heralds a huge potential for determining critical quality attributes (CQAs) in iPSC-derived progenies, and is ideally suited as a quality control tool in cell therapy manufacturing.

### Magnetic Resonance Relaxometry (MRR) T₂ Measurement for Evaluating iPSC Quality Variation at Day 1

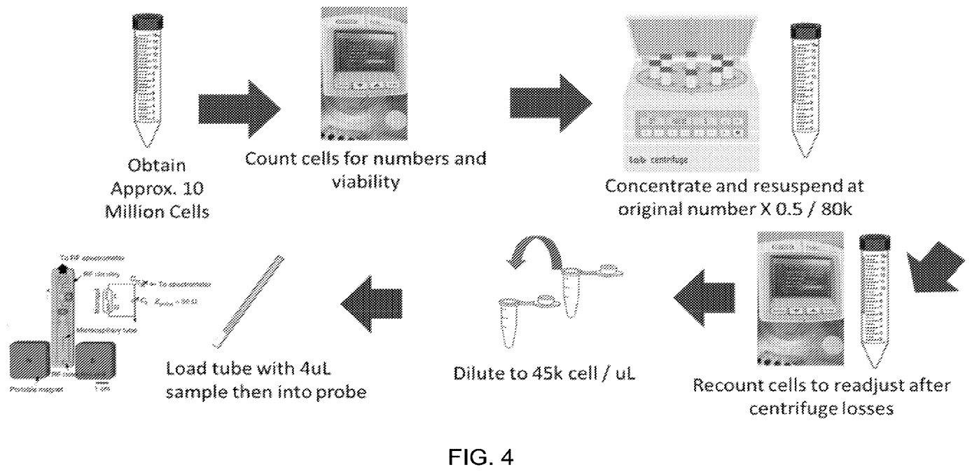

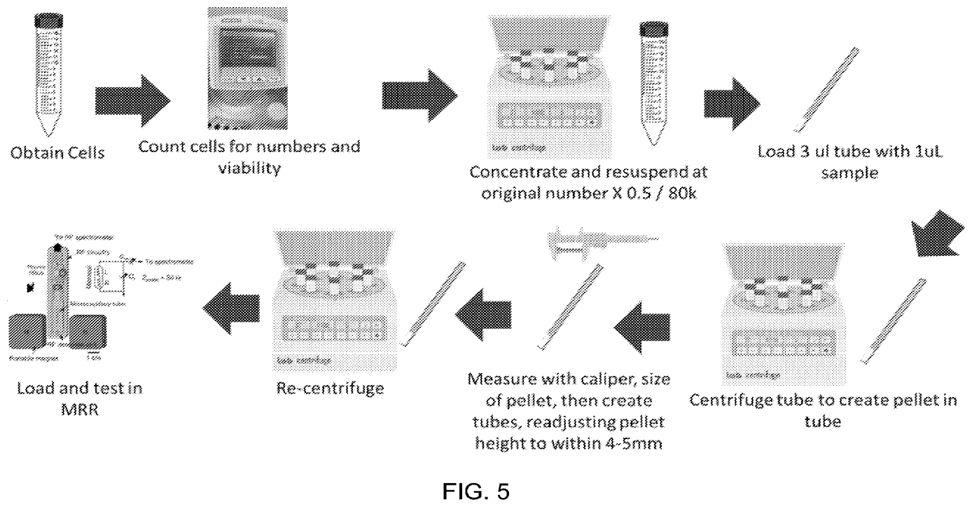

The sample preparation process is shown in both FIG. 4 and FIG. 5. The method shown in FIG. 4 uses suspended cells whilst FIG. 5 shows a method for a pellet of cells. Note that a higher magnetic content results in a lower T₂ relaxation time.

### T₂ Relaxation Times for Batch-to-Batch Induced Pluripotent Stem Cells (iPSCs) Indicating Cell Variability

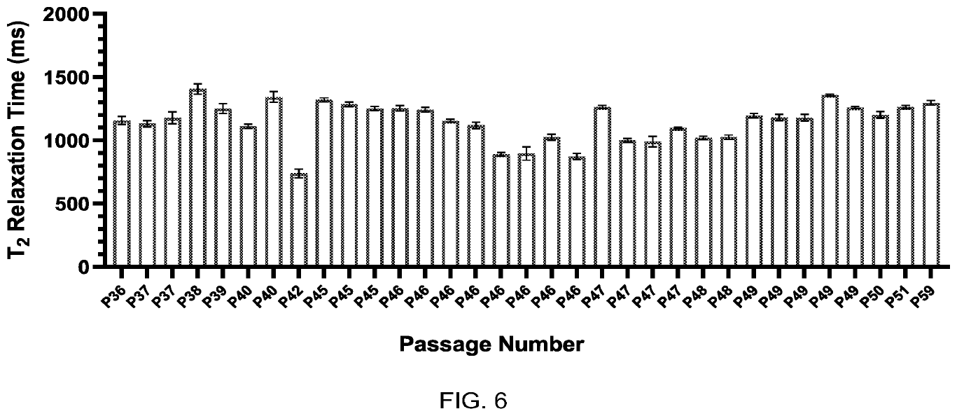

Existing protocols typically grow iPSCs by passaging on a regular basis. The new passage is considered the same as the previous passage. In the past year, several magnetic measurements have been made of the iPSCs at each passage over an extended period of time (+6 months). Shown in FIG. 6, these measurements indicate that contrary to current opinion, iPSCs cultured in labs exhibit huge variability in their Fe³⁺ levels. iPSCs of one passage cannot, therefore, be considered the same as the previous passage, nor perhaps even when they were first extracted.

### T₂ Relaxation Times Under Controlled Single Cell Passage Conditions

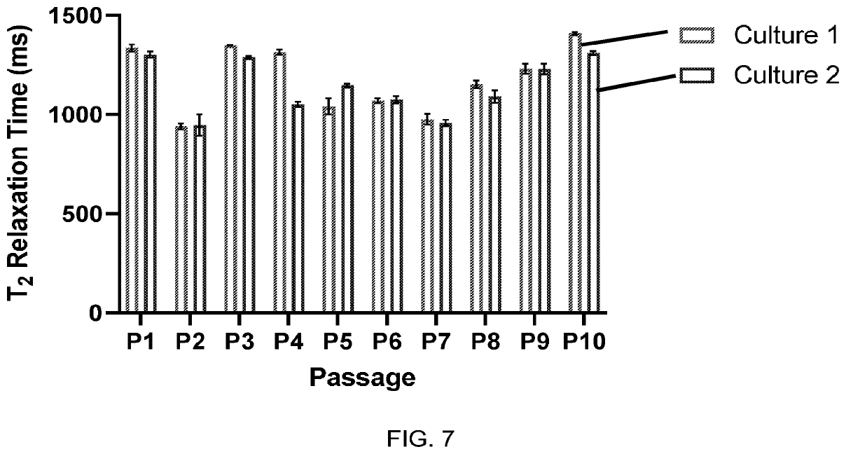

Next, to further understand some of the factors which could lead to such variability, a month-long study was conducted, controlling for parameters such as seeding density, passage time, and plate type passaging in single cells using the MRR method 1. The T₂ relaxation times are shown in FIG. 7 and like in FIG. 6 show variation in the intracellular iron levels, based on T₂ relaxation times. Therefore, it would be challenging to control such a variation in iPSC production, stressing the importance of critical quality attributes (CQA) for such quality variation (e.g., T₂) in cell manufacturing.

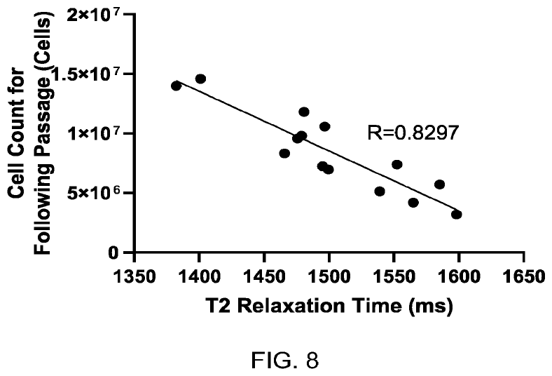

During this experiment, the numbers of cells harvested were also recorded. The data is shown in FIG. 8 and indicated that the T₂ relaxation times for one passage are correlated with the number of cells harvested in the following passage. This suggests that the iron biology of the cells most certainly affects their growth.

### T₂ Relaxation Times Linked to Population Doubling Time

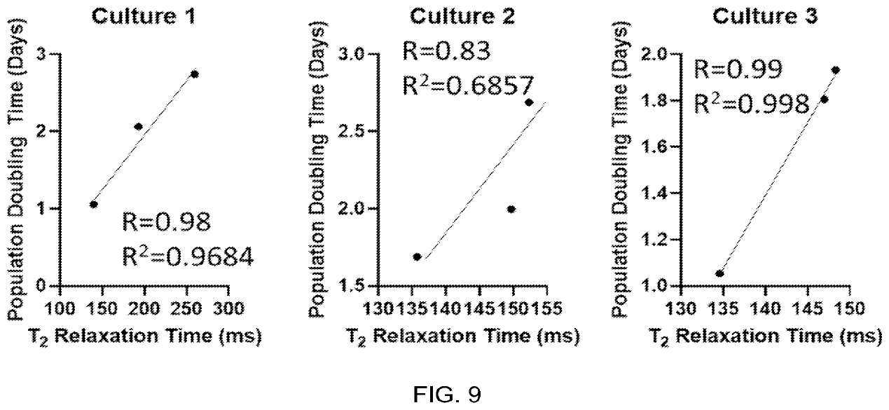

As per FIG. 6, as it was clear that the growth of iPSCs is affected by their iron biology. As another measure of their growth, similar tests were conducted with cells passaged in an alternative method, via clumps. Growing iPSCs via clumps is very common as single cell passaging is known to cause genetic aberrations in iPSCs. See, for example, Garitaonandia, I., et al., 2015. Increased risk of genetic and epigenetic instability in human embryonic stem cells associated with specific culture conditions. PloS one, 2015, 10(2), p. e0118307, which is incorporated by reference in its entirety. iPSC growth was measured via population doubling time, taken from computer analysis of confluency from microscope images. Population data time Vs T₂ Relaxation Time are plotted in FIG. 9. The MRR instrument can determine the population doubling time of a culture.

### Effect of Freeze-Thawing Protocols on iPSCs and T₂ Relaxation Times

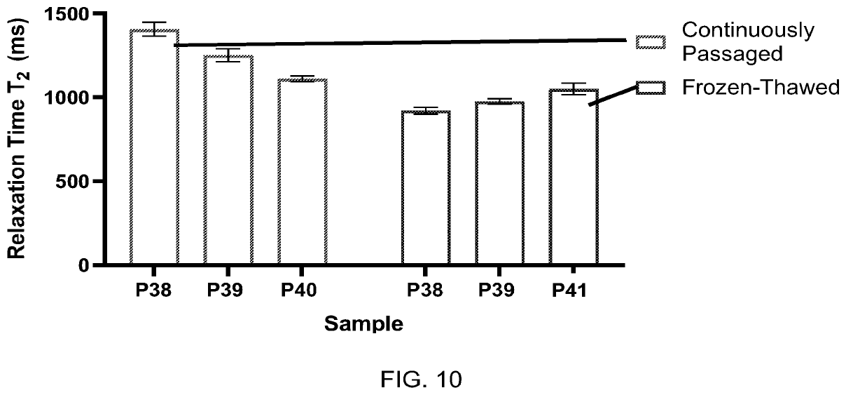

Lastly, aside from passaging of cells, another common laboratory technique to preserve cells is via cryopreservation. While necessary in cell manufacturing, such freeze-thawing processes can impact cell phenotypes and quality significantly. Data for iPSCs that had not been frozen, versus for iPSCs that had been frozen and thawed is shown in FIG. 10. These data indicate that the freeze-thaw process has a significant impact on the cells, in terms of iron phenotypes.

### Large Clumps During Seeding Slow the Population Doubling Time

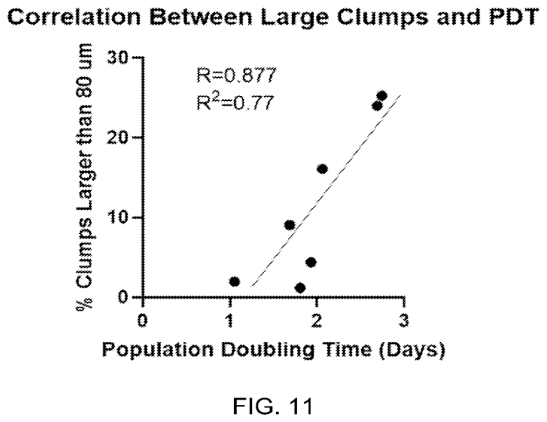

When seeding cells as clumps, it is possible to control the number of clumps being seeded, but not the size of the clumps. Is it commonly known that if the clumps are too small, the cells within them will likely die. However, when the clumps are large, this distribution can be difficult to control. In conjunction with the measurement of T₂ relaxation times, confluency and population doubling time from FIG. 9, the size of the clumps was also determined. In FIG. 11, when the percentage of the cells that is larger than 80 μm increases, the population doubling time also increases. This can be important as fast population doubling times can be linked to healthier iPSCs. See, for example, Ruiz, S., et al., A high proliferation rate is required for cell reprogramming and maintenance of human embryonic stem cell identity. Current Biology, 2011, 21(1), pp.45-52, which is incorporated by reference in its entirety.

Based on the data in FIG. 9 and FIG. 11, it can be an uncontrolled factor by way of clump sizes that influences the variability in iPSC cell culture. Irrespective of whether cell culture is controllable via clump sizes or not, MRR is able to measure their variability passage to passage in a label-free, non-destructive manner. See, for example, Sullivan, S., et al., Quality control guidelines for clinical-grade human induced pluripotent stem cell lines. Regenerative medicine, 2018, 13(7), pp.859-866, which is incorporated by reference in its entirety.

### Correlating Day 1 CQA Measurement with Day 10 SCPC Quality Variation

Spinal cord progenitor cells (SCPCs) are neural progenitor cells derived from induced pluripotent stem cells (iPSCs). However, during this differentiation process, there has been a huge amount of variability in the outcomes of the differentiation, especially in the residual levels of the OCT4 expression. To be able to characterize these ‘impurity’ levels, a correlation between the MRR T₂ relaxation time values of these SCPCs (at Day 10) to their residual OCT4 expression has been found. Different experiments have been conducted to study the SCPC differentiation outcomes on various time points of the process.

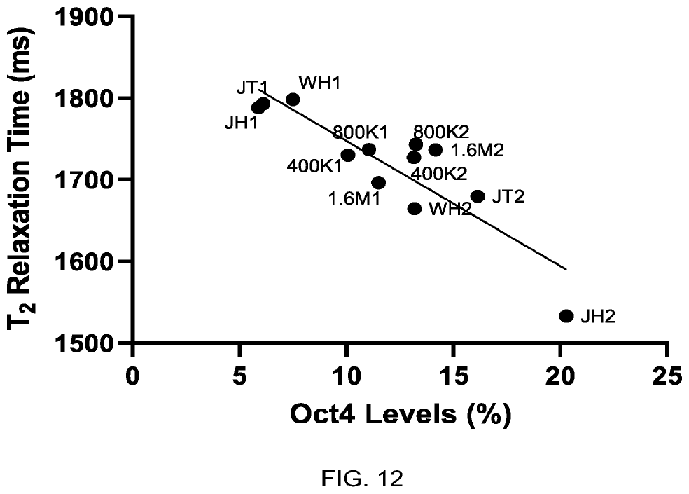

As shown in FIG. 12 below, T₂ values of Day 10 SCPCs is correlated with the percentage of OCT4 positive cells in the culture. Since OCT4+ cells represent cells with limited differentiation, OCT4+ level is a clear quality metric of the produced SCPCs at day 10, just before neural tissue construction and implantation. Therefore, a label-free, rapid analysis of T₂ levels in SCPCs at day 10 can be used as a valid CQA measurement, evaluating the risk of cancer-like tissue generation in the implants.

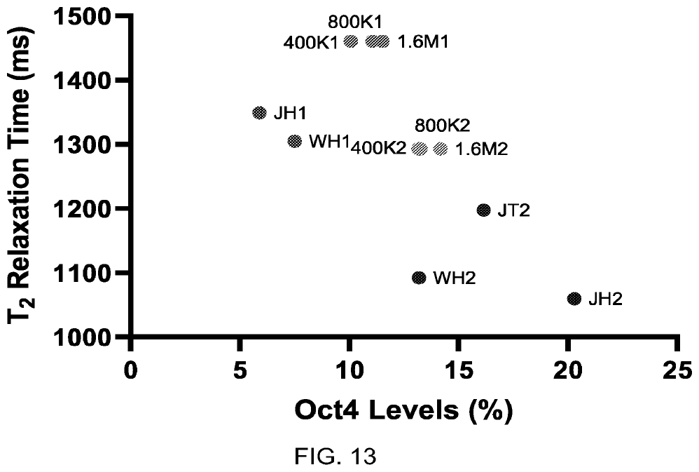

Significantly, the general quality of iPSCs at day 1, quantified by MRR T₂ measurement, can be correlated with the quality of SCPCs (OCT4+percentage) at day 10 (See FIG. 13). This finding can be considered a significant step forward toward truly actionable CQA measurements, implementable as early as Day 1 to predict the eventual likelihood of successful expansion and differentiation into target SCPC and neural stem cells. Given that many known and unknown factors in iPSC expansion lead to significant variation, identifying good and bad batches of cells, as early as possible, can be meaningful for overall cell production and manufacturing workflow.

Other experiments were also performed.

### T₂ Increased as iPSCs Differentiated into SCPCs Across a 10-Day Period

SCPCs were generated from iPSCs via the use of three small molecules that mimic the native development pathway of the spinal cord. See, for example, H. Kumamaru et al., ‘Generation and post-injury integration of human spinal cord neural stem cells’, Nat. Methods, vol. 15, no. 9, Art. no. 9, September 2018 and K. Kajikawa et al., ‘Cell therapy for spinal cord injury by using human iPSC-derived region-specific neural progenitor cells’, Mol. Brain, vol. 13, no. 1, p. 120, December 2020, each of which is incorporated by reference in its entirety. Quantification of cellular phenotypes upon differentiation is usually obtained via FACS or immunocytochemistry staining. Hence, the differentiation efficiency of SCPCs on day 10 was quantified via the expression of pluripotent marker (OCT4) and neural progenitor marker (SOX1).

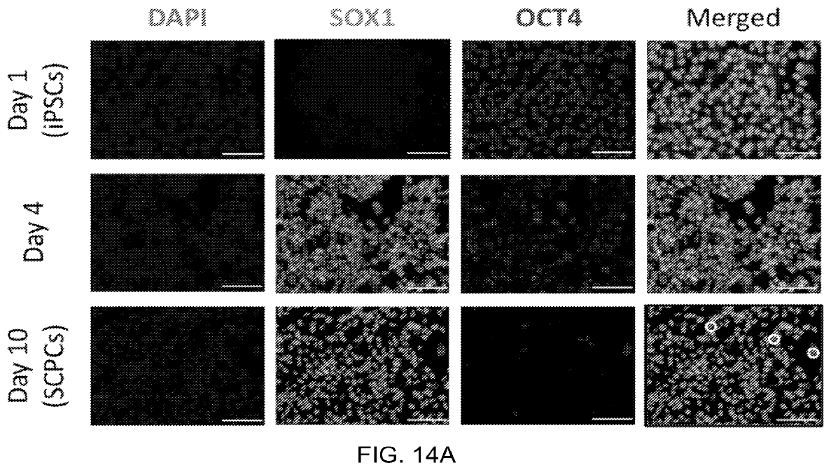

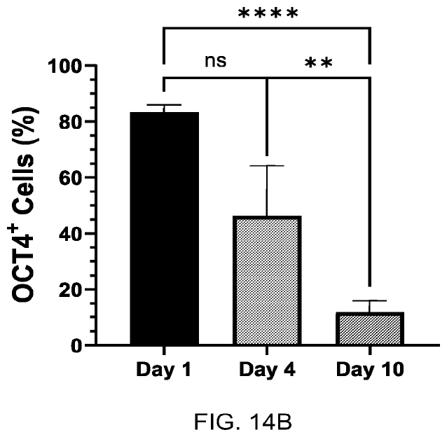

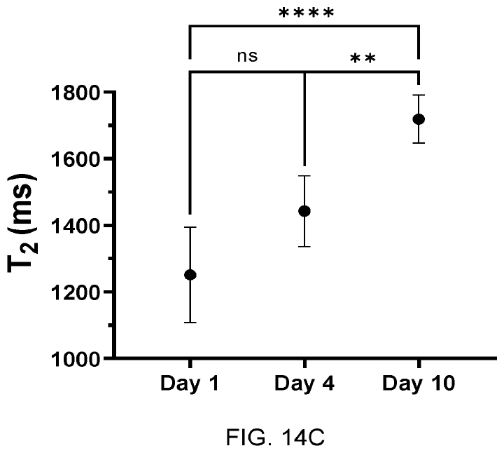

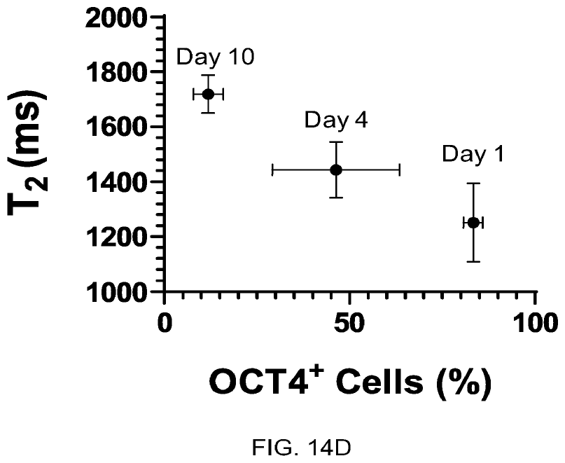

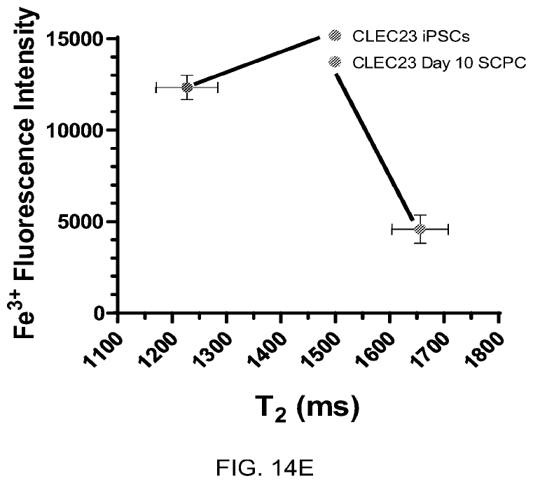

Changes in OCT4 and SOX1 expression were observed as iPSCs differentiated into SCPCs across three different timepoints including day 1 (undifferentiated iPSC), day 4 (mid-point differentiated iPSCs) and day 10 (SCPCs). FIG. 14A illustrates the immunostaining results of OCT4 and SOX1 at day 1, day 4 and day 10. As iPSCs began to differentiate into SCPCs, high expression of SOX1 was observed on day 4 while OCT4 was still decreasing. On the final day of the differentiation, SOX1 was still highly expressed with a relatively low expression of OCT4. This small cell population that expressed OCT4 was typically noted to be residual undifferentiated iPSCs (marked by white circles in day 10 merged image).

A significant increase (p<0.0001) in T₂ was observed as iPSCs differentiated into SCPCs across the 10-day period (FIG. 14B). A quantification of OCT4⁻ cells via immunostaining images showed a significant (p<0.0001) decrease in OCT4⁺ levels as iPSCs differentiated into SCPCs after 10 days (FIG. 14C). T₂ was plotted against OCT4⁺ cells to demonstrate the changes in T₂ as iPSCs differentiate to SCPCs across the 10-day period (FIG. 14D).

A Fe³⁺ stain quantified via FACS validated that T₂ values of iPSCs and SCPCs corresponds to their intracellular Fe³⁺ content (FIG. 14E). iPSCs were shown to have a higher RPE fluorescence mean intensity (i.e., higher Fe³⁺ content) as compared to SCPCs. The presence of artificially spiked iPSCs in the SCPC population decreases measured T₂ values in a dose-dependent manner and exhibits safety risks.

To validate that T₂ values of SCPCs decrease from an increasing presence of iPSCs, SCPCs were artificially spiked with 1-10% of iPSCs. Correspondingly, a significant decrease in T₂ (p<0.0001) was seen as the spiking ratio increased to 5% and beyond (FIG.

15A).

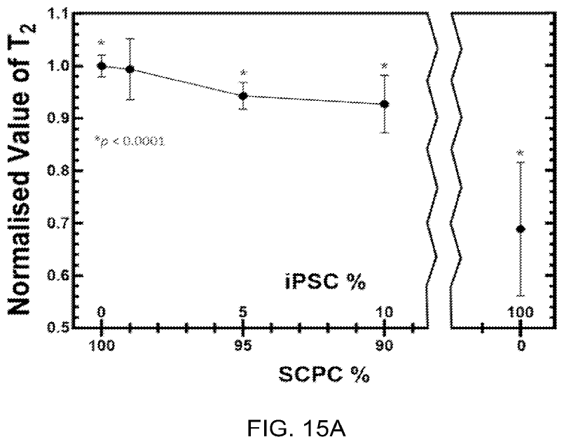

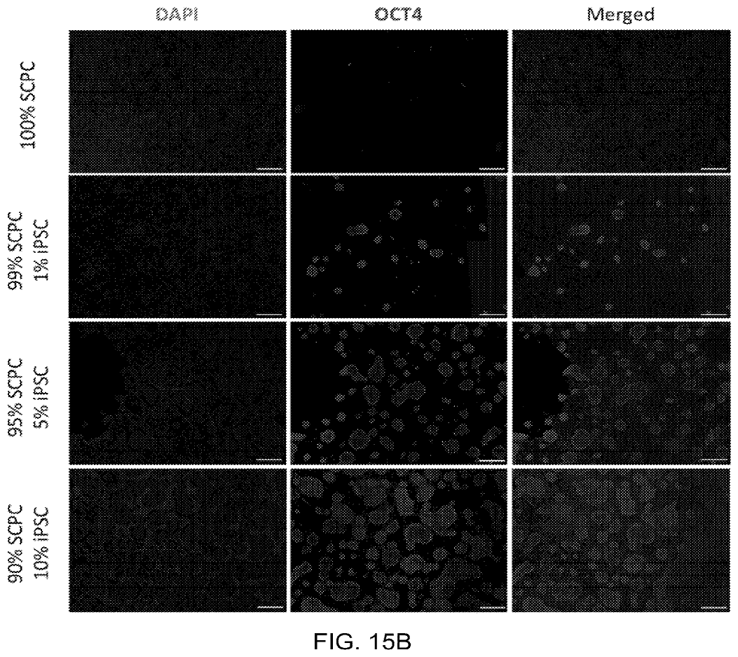

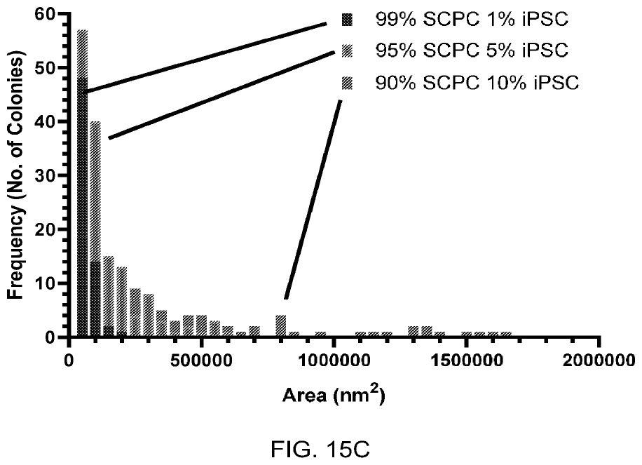

To evaluate the potential safety risks associated with SCPC cultures with lower T₂, the spiked samples were subjected to a colony formation assay. Specifically, by culturing all experimental groups in iPSCs maintenance media for 6 days, an increase in iPSC colonies and colony sizes were observed with increasing amount of spiked iPSCs (FIGS. 15B-15C).

### OCT4 Levels in Day 10 SCPC Population is Correlated with Day 10 T₂ Measurements and Can Be Predicted by T₂ Measurement of Starting iPSCs

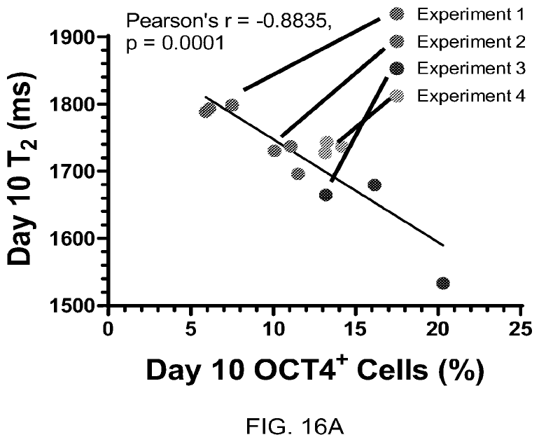

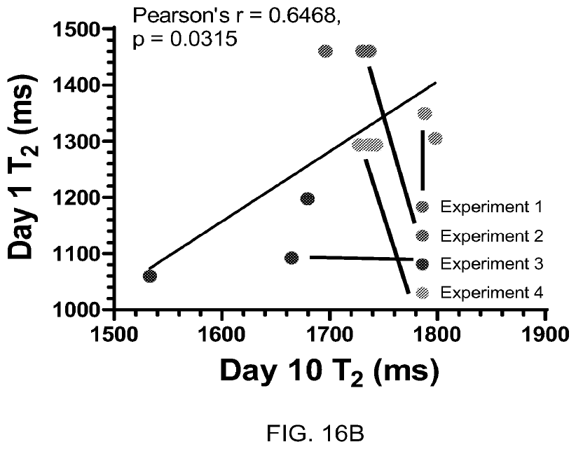

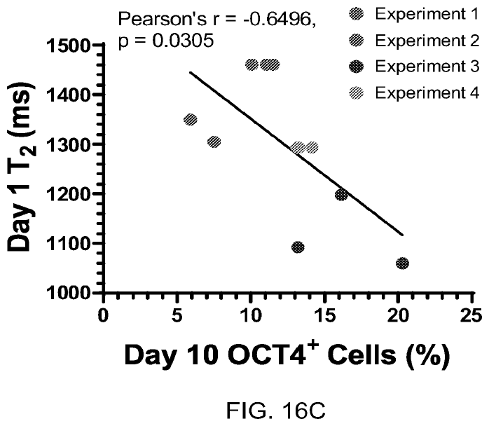

To investigate if MRR was able to detect variations in day 10 residual undifferentiated iPSCs, day 10 T₂ were correlated to the day 10 OCT4⁺ levels. The results indicated that an increased number of OCT4 positive cells in day 10 SCPCs decreased the T₂ measurements in a negative linear correlation trendline (FIG. 16A).

To investigate if day 1 T₂ could provide a prediction of the differentiation outcomes on day 10, several correlations were made. Specifically, day 1 T₂ measurements were correlated to both day 10 T₂ measurements and day 10 OCT4⁺ levels. The correlations clearly indicated that lower T₂ of day 1 iPSCs resulted in lower day 10 SCPC T₂ and higher day 10 OCT4⁺ levels. (FIGS. 16B-16C).

### T₂ Decreases in Accordance with Reduced Levels of Neural Stem and Progenitor Cell Markers

To evaluate the efficacy of MRR in reflecting the quality of SCPCs, the extent of SCPC differentiation was modulated by reducing CHIR-99021 concentration in the neural differentiation media. CHIR-99021 is a potent GSK3 inhibitor responsible for the level of activation of the Wnt signaling pathway that controls the differentiation efficiency of iPSCs to various progenitor cell types. See, for example, Z.-W. Du et al., ‘Generation and expansion of highly pure motor neuron progenitors from human pluripotent stem cells’, Nat. Commumn., vol. 6, no. 1, Art. no. 1, March 2015 and T. Qian, T. M. Heaster, A. R. Houghtaling, K. Sun, K. Samimi, and M. C. Skala, ‘Label-free imaging for quality control of cardiomyocyte differentiation’, Nat. Commun., vol. 12, no. 1, p. 4580, December 2021, each of which is incorporated by reference in its entirety.

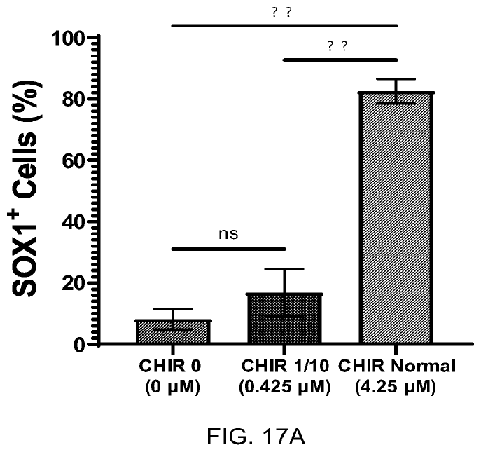

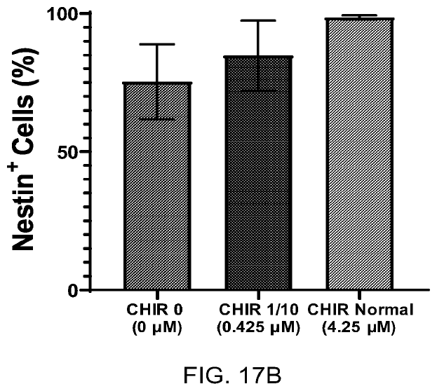

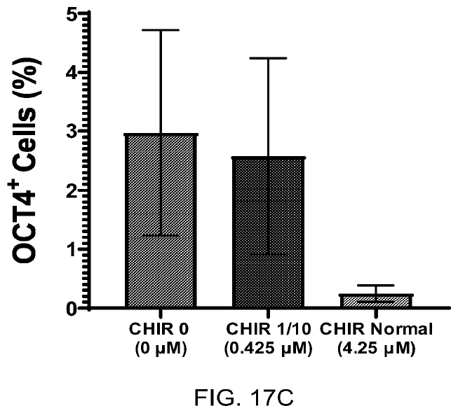

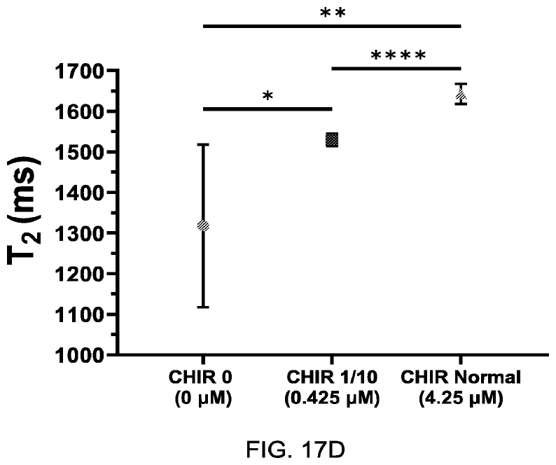

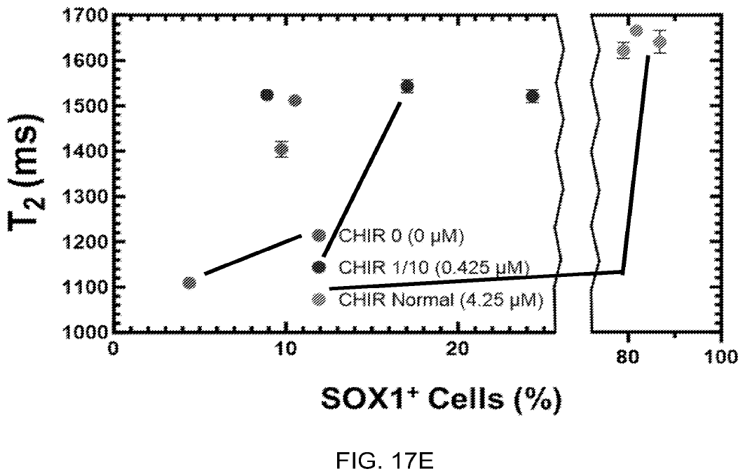

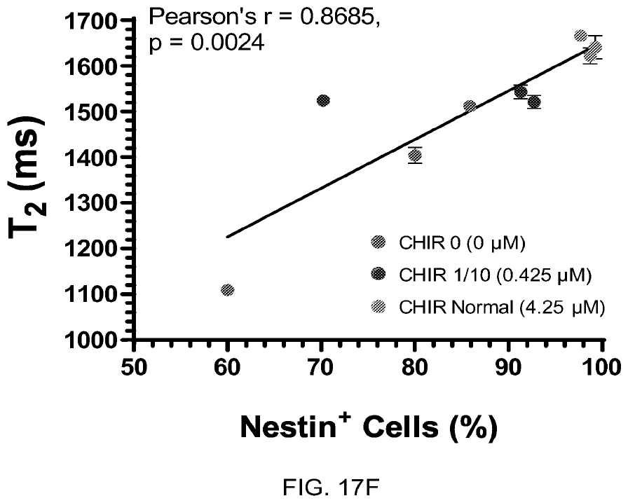

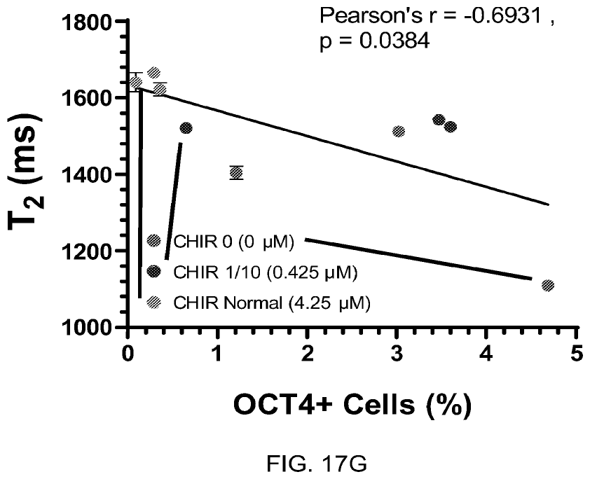

Experimental groups included an absence of CHIR-99021 (CHIR 0, 0 μM), 1/10 lower concentration of CHIR-99021 (CHIR 1/10, 0.425 μM) and the control group (CHIR Normal, 4.25 μM). Decreasing CHIR concentration was shown to affect the levels of SOX1⁺, Nestin⁺ (neural stem cell marker) and OCT4⁺ cells in day 10 SCPCs (FIGS. 17A-17C).

More importantly, T₂ was significantly lesser in CHIR 0 (p<0.01) and CHIR 1/10 (p<0.0001) as compared to CHIR Normal (FIG. 17D). T₂ also corresponded to the decrease in SOX1 and Nestin expression and the increase in OCT4 expression in both CHIR 0 and CHIR 1/10as compared to CHIR Normal (FIGS. 17E-17G).

### Validation on Different iPS Cell Lines: CLEC23 and BJ iPSCs

To establish and validate the robustness of MRR in identifying the differentiation efficiency of iPSCs to SCPCs, another iPSC line was employed to validate the initial findings. Specifically, the use of BJ-iPSC cell line was meant to represent fibroblast-derived iPSCs that were previously used to generate spinal motor neurons. See, for example, S.-Y. Ng et al., ‘Genome-wide RNA-Seq of Human Motor Neurons Implicates Selective ER Stress Activation in Spinal Muscular Atrophy’, Cell Stem Cell, vol. 17, no. 5, pp. 569-584, November 2015 and J.-H. Hor et al., ‘ALS motor neurons exhibit hallmark metabolic defects that are rescued by SIRT3 activation’, Cell Death Differ., vol. 28, no. 4, Art. no. 4, April 2021, each of which is incorporated by reference in its entirety.

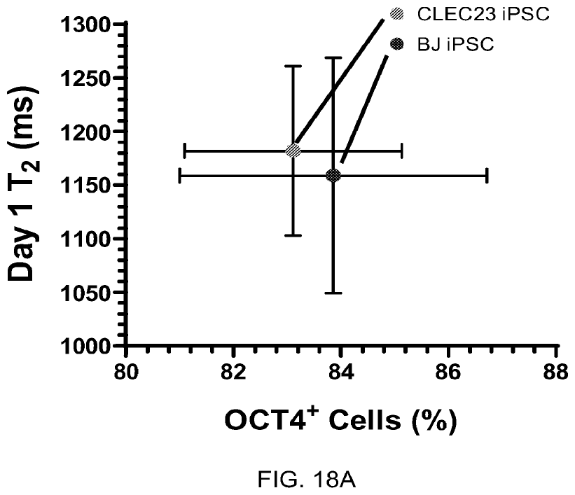

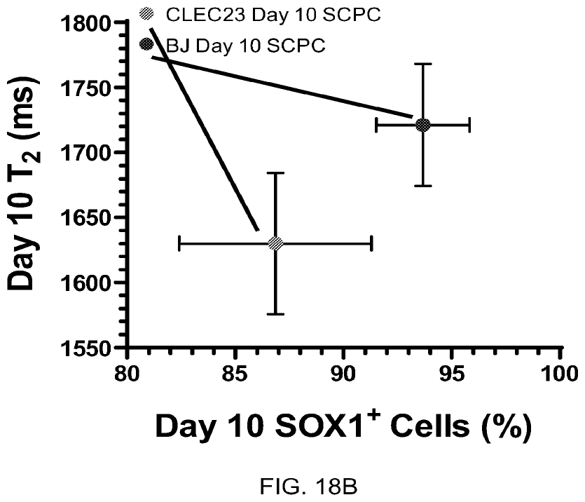

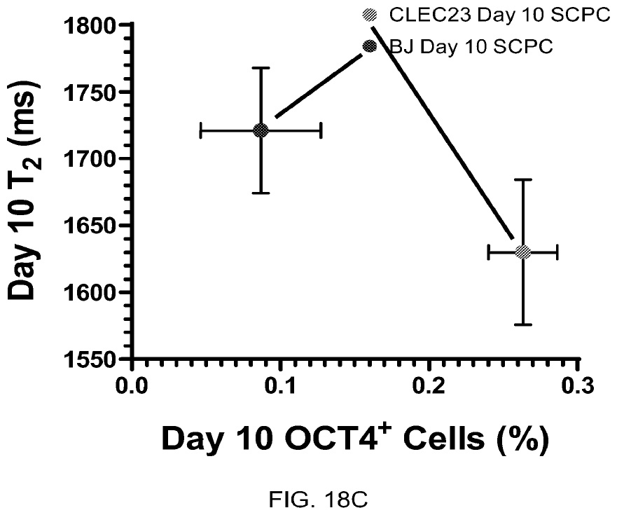

The T₂ and OCT4 levels of BJ-iPSCs were of similar ranges with CLEC23 iPSCs (FIG. 18A). However, T₂ of day 10 BJ-SCPCs was generally higher as compared to day 10 CLEC23 SCPCs (FIGS. 18B-18C). The T₂ of day-10 BJ SCPCs corresponded to its SOX1 and OCT4 expression which were respectively higher and lower as compared to CLEC23 SCPCs.

Detection of cells with the potential for tumorigenicity and ensuring the quality of the progenitor cells used for regenerative cell therapy is a significant bottleneck. For example, a recent clinical trial for iPSC-derived islet beta cell therapy for diabetes resulted in teratoma formation two months post-transplantation. See, for example, L. Han et al., ‘Distinctive Clinical and Pathologic Features of Immature Teratomas Arising from Induced Pluripotent Stem Cell-Derived Beta Cell Injection in a Diabetes Patient’, Stem Cells Dev., vol. 31, no. 5-6, pp. 97-101, March 2022, which is incorporated by reference in its entirety. Furthermore, the tumor was characterized as an immature teratoma and expressed both OCT4 and SOX2 in immunofluorescence assays. Nori and colleagues attempted to implant iPSC-derived neural progenitors into SCI mouse models, with initial functional recovery and synapse formation 47 days post-transplantation. See, for example, S. Nori et al., ‘Long-Term Safety Issues of iPSC-Based Cell Therapy in a Spinal Cord Injury Model: Oncogenic Transformation with Epithelial-Mesenchymal Transition’, Stem Cell Rep., vol. 4, no. 3, pp. 360-373, March 2015, which is incorporated by reference in its entirety. However, when the study was extended to 103 days or more, they observed the formation of tumors that led to a reduction in motor function. In these tumors, the OCT4 transgene was found to be activated and had a significant presence of undifferentiated Nestin⁺ neural cells. A recent clinical trial that transplanted iPSC-derived neural stem and progenitor cells also determined the presence of residual undifferentiated iPSCs by quantifying the expression of OCT4 in the cell population. See, for example, K. Sugai et al., ‘First-in-human clinical trial of transplantation of iPSC-derived NS/PCs in subacute complete spinal cord injury: Study protocol’, Regen. Ther., vol. 18, pp. 321-333, December 2021, which is incorporated by reference in its entirety. Hence, detecting OCT4⁺ cells in iPSC-derived progenies is critical for ensuring and validating the safety of iPSC-related cell therapy products.

The current assays to quantify phenotypes of iPSCs and progenitor cells employ label-based fluorescent markers, which are analyzed via a flow cytometer or visualized via a fluorescent microscope. See, for example, T. Kuroda et al., ‘Highly Sensitive In Vitro Methods for Detection of Residual Undifferentiated Cells in Retinal Pigment Epithelial Cells Derived from Human iPS Cells’, PLOS ONE, vol. 7, no. 5, p. e37342, May 2012, which is incorporated by reference in its entirety. However, these assays often perturb or destroy the cells from terminal fixation, rendering them unusable for subsequent applications. Furthermore, these assays are expensive, laborious, and time-consuming, ultimately inadequate for quality control of iPSCs or iPSC-derived progenies in bioproduction processes. Biochemical, omics-based methods are the ultimate standard of determining phenotypic characteristics of the cells. Still, most of them are population-based assays, requiring averaging the cell signalling over a population of cells. Given the common heterogeneity of the stem cells and their progenitors, they are generally not adequate to monitor the presence of low-abundance cells with safety risks.

On the other hand, label-free technologies that can characterize cellular biophysical characteristics, which correlate well to cell quality and safety, allows rapid and non-destructive assessment of cell batch quality, ideally suited for large-scale cell manufacturing. In this work, a rapid and non-destructive method to validate safety and quality phenotypes for iPSC-derived SCPCs generated in vitro is described.

T₂ readouts via MRR was shown to correspond with the phenotypic transition from iPSCs to SCPCs, with iPSCs exhibiting more paramagnetic contents (higher intracellular Fe³⁺) in general (FIGS. 14A-14E). An Fe³⁺ stain analysed via FACS also demonstrated that T₂ measurements of iPSCs and SCPCs corroborate to its Fe³⁺ levels (FIG. 14E). These findings clearly indicates that T₂ measurements are a good surrogate for the label-free measurement of intracellular Fe³⁺ content. The difference between the iron content of iPSCs and SCPCs was essential in developing a hypothesis to detect the purity of the SCPC population after its differentiation process. Thus, OCT4 levels in SCPCs can be measured via the MRR system and was investigated. This measurement could provide an endpoint assessment of differentiation efficiencies in SCPC batch-to-batch production.

Spiking of iPSCs demonstrated that various percentages of iPSCs can be estimated using the MRR system (FIG. 15A). Further demonstration of safety risks in SCPCs associated with lower T₂ values (due to an increasing presence of residual undifferentiated iPSCs) was done via a colony culture assay. Colony formation assays were previously used to evaluate and determine the presence of any residual undifferentiated iPSCs in differentiated cells. See, for example, K. Tano, S. Yasuda, T. Kuroda, H. Saito, A. Umezawa, and Y. Sato, ‘A Novel In Vitro Method for Detecting Undifferentiated Human Pluripotent Stem Cells as Impurities in Cell Therapy Products Using a Highly Efficient Culture System’, PLOS ONE, vol. 9, no. 10, p. e110496, October 2014 and T. Watanabe et al., ‘Multisite studies for validation and improvement of a highly efficient culture assay for detection of undifferentiated human pluripotent stem cells intermingled in cell therapy products’, Cytotherapy, vol. 23, no. 2, pp. 176-183, February 2021, each of which is incorporated by reference in its entirety. However, this study employed this assay for an illustration of the safety issues associated with low T₂ values. The results of the spiking experiment indicated that increasing amounts of iPSCs within SCPC populations led to proportional decrement of T₂. However, these results were insufficient in concluding that MRR can be used to detect residual undifferentiated iPSCs in an actual day-10 SCPC population. Firstly, T₂ of iPSCs consists of significant batch-to-batch variability which caused some of the spiking experiments to have larger decrements of T₂ as compared to others. Secondly, there was a possibility that residual undifferentiated iPSCs might not exhibit the same T₂ as day 1 undifferentiated iPSCs. Hence, a closer look at day 10 SCPCs was taken and made several different correlations to demonstrate MRR capabilities in identifying variations of residual undifferentiated iPSCs.

Several batches of day 10 CLEC23 SCPCs were evaluated on the number of their residual undifferentiated iPSCs via immunofluorescence staining, and we correlated the T₂ to OCT4⁺ cell percentages in day 10 SCPCs (FIG. 16A). This phenomenon occurred because an SCPC population with higher numbers of residual undifferentiated iPSCs effectively contributes to the total amount of iron content present within the sample. This effectively reduces the values of T₂ as compared to SCPCs with lesser residual undifferentiated iPSCs. Furthermore, iPSCs with lower T₂ were shown to indicate lower T₂ and higher OCT4 levels after differentiating to SCPCs (FIGS. 16B-16C). This indicates that T₂ measurements of iPSCs, as early as day 1 in the differentiation process, may be used to predict the differentiation efficiency outcome of iPSCs to SCPCs. This predictive ability is highly beneficial to determine if a batch of iPSCs would result in a high-quality batch of SCPCs. Such an early-stage quality assessment tool would be highly desirable in iPSC manufacturing workflow, which may suffer from significant donor-to-donor and batch-to-batch quality variations.

Even though the lack of undifferentiated iPSCs within SCPC populations, measured by MRR, could serve as a safety CQA, it does not automatically translate into high-quality SCPCs. To establish the feasibility of MRR as an indicator of quality, a negative disruption of the differentiation efficiency was done to obtain the T₂ of poorly differentiated SCPCs. Disruption of the differentiation via lowering CHIR concentration resulted in having lower SOX1⁺ and Nestin⁺ cells and higher OCT4⁺ cells (FIGS. 17A-17C). Based on the previous findings, it strongly suggests that the increased presence of OCT4⁺ cells also contributed to the decrease in T₂ of poorly differentiated SCPCs. These findings demonstrated that T₂ measurements strongly corresponded to poor SCPC differentiation outcomes (FIGS. 17D-17G). Again, these results confirm that T₂ of a differentiated SCPC population can indicate if the differentiation was of poor quality.

Another iPSC line (BJ-iPSCs) was employed to demonstrate that T₂ values of iPSCs and SCPCs was not innate to the CLEC23 iPSC line. Differences in the differentiation outcomes of CLEC23 and BJ iPSCs to SCPCs were. The percentage of SOX1⁺ and OCT4⁺ cells were found to be higher and lower, respectively, in day 10 BJ-SCPCs than day 10 CLEC23 SCPCs. In correspondence to the phenotypes, T₂ measurements of day 10 BJ-SCPCs were also found to be higher than CLEC23 SCPCs (FIGS. 18B-18C). This strongly suggests that higher T₂ values do correspond to a higher differentiation efficiency of iPSCs to SCPCs. This indicates that T₂ can be employed to assess the differentiation potential of other iPSC lines in differentiating to SCPCs.

Currently, the exact mechanistic relationship between intracellular iron level and cellular phenotype (such as stem cells, progenitors, and fully differentiated cells) is poorly understood, even though there has been an explosion of scientific interest in the topic recently. Previously, it was reported magnetic resonance imaging (MRI) was employed to track the in vitro differentiation of MSCs to neural cells by coupling the ferritin gene to neural cell specific promoters. See, for example, C. Song et al., ‘Use of Ferritin Expression, Regulated by Neural Cell-Specific Promoters in Human Adipose Tissue-Derived Mesenchymal Stem Cells, to Monitor Differentiation with Magnetic Resonance Imaging In Vitro’, PLOS ONE, vol. 10, no. 7, p. e0132480, July 2015, which is incorporated by reference in its entirety. Furthermore, differentiation outcomes of iPSCs to various progenitors have been enhanced with iron treatment or the addition of iron-binding proteins. See, for example, D. Lu et al., ‘Accelerated Neuronal Differentiation Toward Motor Neuron Lineage from Human Embryonic Stem Cell Line (H9)’, Tissue Eng. Part C Methods, vol. 21, no. 3, pp. 242-252, March 2015 and F. Zhang et al., ‘Transferrin improved the generation of cardiomyocyte from human pluripotent stem cells for myocardial infarction repair’, J. Mol. Histol., vol. 52, no. 1, pp. 87-99, February 2021, each of which is incorporated by reference in its entirety. Yet, there exists no studies that has elucidated the changes in intracellular iron content as one cell type differentiates to another. Still, our data demonstrates that MRR T₂ are a non-destructive and label-free endpoint assay to rapidly measure the differentiation efficiency of iPSC-derived neural stem and progenitor cell phenotypes for rapid validation of safety and quality parameters.

This is the first reported study that utilised MRR to identify differences in cell types via their intracellular Fe³⁺ content. However, the use of label-free technologies to provide an indication or analysis of cellular phenotypes had been previously demonstrated by Qian et al., who implemented an autofluorescence system to quantify the differentiation efficiency of iPSC-derived cardiac progenitors. See for example, T. Qian, T. M. Heaster, A. R. Houghtaling, K. Sun, K. Samimi, and M. C. Skala, ‘Label-free imaging for quality control of cardiomyocyte differentiation’, Nat. Commun., vol. 12, no. 1, p. 4580, December 2021, each of which is incorporated by reference in its entirety. Autofluorescence from endogenous fluorophores such as NADH and FAD are involved in the generic energy metabolism of cells, which suggests that stem cells and their progenitors are of dissimilar energy phenotypes. See, for example, D. Lu et al., ‘Accelerated Neuronal Differentiation Toward Motor Neuron Lineage from Human Embryonic Stem Cell Line (H9)’, Tissue Eng. Part C Methods, vol. 21, no. 3, pp. 242-252, March 2015, which is incorporated by reference in its entirety. Quantifying intracellular Fe³⁺ paramagnetic ions, which are related to the ‘stored iron’ but not the ‘active iron’ (Fe²⁺) within the cells, was determined to be an indicator for the generic cell metabolic activity.

The iron content of cells recently gained a lot of interest among researchers, with potential implications in many physiological and pathophysiological processes such as aging, activation, cellular senescence, cancer, and inflammation. See, for example, W. J. Chen, G. P. Kung, and J. P. Gnana-Prakasam, ‘Role of Iron in Aging Related Diseases’, Antioxidants, vol. 11, no. 5, p. 865, April 2022, T. Sato, J. S. Shapiro, H.-C. Chang, R. A. Miller, and H. Ardehali, ‘Aging is associated with increased brain iron through cortex-derived hepcidin expression’, eLife, vol. 11, p. e73456, January 2022, S. Kuvibidila, R. P. Warrier, and B. Surendra Baliga, ‘An overview of the role of iron in T cell activation’, J. Trace Elem. Exp. Med., vol. 16, no. 4, pp. 219-225, 2003, S. Ni, Y. Yuan, Y. Kuang, and X. Li, ‘Iron Metabolism and Immune Regulation’, Front. Immunol., vol. 13, 2022, Accessed: Jun, 12, 2023, D. W. Killilea, S. L. Wong, H. S. Cahaya, H. Atamna, and B. N. Ames, ‘Iron Accumulation during Cellular Senescence’, Ann. N. Y. Acad. Sci., vol. 1019, no. 1, pp. 365-367, June 2004, A. Cozzi et al., ‘Stem Cell Modeling of Neuroferritinopathy Reveals Iron as a Determinant of Senescence and Ferroptosis during Neuronal Aging’, Stem Cell Rep., vol. 13, no. 5, pp. 832-846, November 2019, D. H. Manz, N. L. Blanchette, B. T. Paul, F. M. Torti, and S. V. Torti, ‘Iron and cancer: recent insights’, Ann. N. Y. Acad. Sci., vol. 1368, no. 1, pp. 149-161, March 2016, Q. Guo et al., ‘The Role of Iron in Cancer Progression’, Front. Oncol., vol. 11, 2021, Accessed: Jun. 12, 2023, T. Basak and R. K. Kanwar, ‘Iron imbalance in cancer: Intersection of deficiency and overload’, Cancer Med., vol. 11, no. 20, pp. 3837-3853, 2022, M. Wessling-Resnick, ‘Iron Homeostasis and the Inflammatory Response’, Annu. Rev. Nutr., vol. 30, pp. 105-122, August 2010, D. B. Kell and E. Pretorius, ‘Serum ferritin is an important inflammatory disease marker, as it is mainly a leakage product from damaged cells’, Metallomics, vol. 6, no. 4, pp. 748-773, April 2014, and O. Marques, G. Weiss, and M. U. Muckenthaler, ‘The role of iron in chronic inflammatory diseases: from mechanisms to treatment options in anemia of inflammation’, Blood, vol. 140, no. 19, pp. 2011-2023, November 2022, each of which is incorporated by reference in its entirety. Yet, there is no systematic understanding regarding the exact roles of Fe²⁺/Fe³⁺ in these critical biological contexts. This gap in knowledge is further exacerbated by difficulties in obtaining accurate measurements of Fe²⁺ and Fe³⁺. The conventional assays and methods for detecting Fe²⁺, Fe³⁺ and even total iron concentration, are largely inaccurate due to the nature of iron ions, inaccurate correlations and the limitation of current technologies. See, for example, T. Hirayama and H. Nagasawa, ‘Chemical tools for detecting Fe ions’, J. Clin. Biochem. Nutr., vol. 60, no. 1, pp. 39-48, January 2017, M. Tenopoulou, T. Kurz, P.-T. Doulias, D. Galaris, and U. T. Brunk, ‘Does the calcein-AM method assay the total cellular “labile iron pool” or only a fraction of it?’, Biochem. J., vol. 403, no. Pt 2, pp. 261-266, April 2007, U. Abbasi, S. Abbina, A. Gill, V. Bhagat, and J. N. Kizhakkedathu, ‘A facile colorimetric method for the quantification of labile iron pool and total iron in cells and tissue specimens’, Sci. Rep., vol. 11, p. 6008, March 2021, J. S. Rohrer, M. S. Joo, E. Dartyge, D. E. Sayers, A. Fontaine, and E. C. Theil, ‘Stabilization of iron in a ferrous form by ferritin. A study using dispersive and conventional x-ray absorption spectroscopy.’, J. Biol. Chem., vol. 262, no. 28, pp. 13385-13387, October 1987, M. A. Knovich, J. A. Storey, L. G. Coffman, and S. V. Torti, ‘Ferritin for the Clinician’, Blood Rev., vol. 23, no. 3, pp. 95-104, May 2009, V. Fiorito, S. Geninatti Crich, L. Silengo, F. Altruda, S. Aime, and E. Tolosano, ‘Assessment of iron absorption in mice by ICP-MS measurements of (57)Fe levels’, Eur. J. Nutr., vol. 51, no. 7, pp. 783-789, October 2012, B. Ar1, S. Z. Can, and S. Bak1rdere, ‘Traceable and accurate quantification of iron in seawater using isotope dilution calibration strategies by triple quadrupole ICP-MS/MS: Characterization measurements of iron in a candidate seawater CRM’, Talanta, vol. 209, p. 120503, March 2020, and C. Song et al., ‘Use of Ferritin Expression, Regulated by Neural Cell-Specific Promoters in Human Adipose Tissue-Derived Mesenchymal Stem Cells, to Monitor Differentiation with Magnetic Resonance Imaging In Vitro’, PLOS ONE, vol. 10, no. 7, p. e0132480, July 2015, each of which is incorporated by reference in its entirety, Moreover, these methods usually require additional reagents or kits for iron measurements, which ultimately subjects the biological samples to further chemical or biological processing.

As the MRR system measures the T₂ of live cells, it provides a unique window to the iron biology of cells. T₂ measurements of iPSCs and SCPCs in this study were well correlated to markers that require terminal fixation for analysis (e.g., OCT4, SOX1, Nestin). Moreover, this method requires a small number of cells (<2×10⁵) and does not require any chemical or biological processing allowing the same cells to be subjected to subsequent biological or functional measurements that can be compared or correlated to their T₂ values. This was demonstrated in previous works where MRR was used to indicate MSC quality by quantifying their senescence levels, which has a direct effect on its downstream therapeutic efficacy. See, for example, S. S. Thamarath et al., ‘Rapid and Live-Cell Detection of Senescence in Mesenchymal Stem Cells by Micro Magnetic Resonance Relaxometry’, Stem Cells Transl. Med., p. szad014, March 2023, which is incorporated by reference in its entirety.

It is important to note that MRR is not without its limitations. Although MRR is reported to be an indicator of quality via T₂, it is important to note that cell phenotypes are not always a direct correlation to subsequent downstream functionalities.

It is also essential to note that variations in differentiation efficiency may likely stem from the initial quality of iPSCs. Recent publications have suggested that iron overload or underload conditions in iPSCs can severely impact the maintenance of their pluripotency. See, for example, Z. Han et al., ‘Iron Homeostasis Determines Fate of Human Pluripotent Stem Cells Via Glycerophospholipids-Epigenetic Circuit’, Stem Cells, vol. 37, no. 4, pp. 489-503, April 2019, and Z. Han et al., ‘Iron overload inhibits self-renewal of human pluripotent stem cells via DNA damage and generation of reactive oxygen species’, FEBS Open Bio, vol. 10, no. 5, pp. 726-733, May 2020, each of which is incorporated by reference in its entirety. Therefore, this recent finding presents a huge potential for MRR to elucidate further insights into the iron homeostasis of iPSCs for quality control purposes. Also, recent data has shown that mechanical stresses such as vibration have significant downstream impacts on the maintenance of iPSCs. See, for example, K. Kanie et al., ‘Effect of mechanical vibration stress in cell culture on human induced pluripotent stem cells’, Regen. Ther., vol. 12, pp. 27-35, December 2019, which is incorporated by reference in its entirety. Another future direction of MRR is to combine the Fe³⁺ measurements with other label-free technologies to provide a multi-variate analysis of cell quality. As MRR provides information on cell quality via a single biophysical characteristic, the combination of Fe³⁺ measurements with other biophysical factors such as cell size, impedance, and autofluorescence will provide a deeper and more robust analysis of cell quality. We believe such a multi-variate analysis will be of significant benefits and a game changer to the cell therapy manufacturing industry.

Using intracellular Fe³⁺ to quantify iPSC and SCPC phenotypes, capabilities of a label-free MRR technology has been demonstrated to perform rapid and non-destructive endpoint assessment of the safety and quality of SCPCs. T₂ measurements were shown to correlate well with conventional biochemical assays of iPSC and SCPC phenotyping. This indicates that T₂ measurements via the MRR system is ideally suited to monitor and assess the safety and quality of iPSC-derived SCPCs produced for spinal cord injury cell therapy applications.

The following describes the methods and the results of the disclosure.

### Materials and Methods

### MRR Measurement

MRR consists of a portable, permanent magnet (Metrolab Instruments, Plan-les-Ouates, Switzerland) with B₀=0.5 T and a bench-top type NMR console (Kea Magritek, Wellington, New Zealand). ¹H MRR measurements were performed at the resonance frequency of 21.65 MHz inside the magnet. A single resonance proton MRR probe with a detection micro-coil of 900-μm inner diameter was used for accommodating the MRR samples into the microcapillary tubes (o.d.: 1,500 μm, i.d.: 950 μm) (22-260-950, Fisherbrand, Waltham, MA, USA). In the MRR probe, the electronic parts and coil were mounted on the single printed circuit board (FIG. 1A). All the experiments were performed at 26.3 °C inside the magnet maintained by a temperature controller (RS component, UK).

For all MRR experiments, the normalized concentration (6×104 cells in 4 μL volume) of cells can be used unless otherwise stated. The cell samples can be spun down at 400 g for 5 minutes, and the supernatant was aspirated. The pellet can be suspended in PBS with concentration of 1.5×104 cells per μL and 4 μL was filled at a 4 mm length of the micro-capillary tube. The micro-capillary tube can be sealed with crystoseal (Leica Microsystems) and mounted into the coil for MRR measurements. Proton transverse relaxation times (T₂) can be measured by standard Carr-Purcell-Meiboom-Gill (CPMG) pulse programme (FIGS. 3A-3B). The transmitter power output can be maintained at 12.5 mW for a single 90° pulse of pulse length 6 μs for all the T₂ measurements. The CPMG train of pulses with inter echo time of 200 μs with 4000 echoes can be used for all experiments. A recycle delay of 2 s, which is sufficient to allow all the spins to return to thermal equilibrium, can be used. For example, twenty-four scans can be performed for experiments for signal averaging.

iPSC Culture

Two human iPSC lines were routinely cultured and maintained in this study on Matrigel (83.3 μg/mL, Corning, USA) coated tissue culture plastic dishes in StemMACS™ iPS-Brew XF (Miltenyi Biotech, Germany). (1) Healthy umbilical cord-lining epithelial cell (CLEC23)-derived iPSCs were kindly provided by Dr Kah-Leong Lim and CellResearch Corporation Pte Ltd. See, for example, Y. Zhou et al., ‘Characterization of Human Umbilical Cord Lining-Derived Epithelial Cells and Transplantation Potential’, Cell Transplant., vol. 20, no. 11-12, pp. 1827-1841, December 2011, each of which is incorporated by reference in its entirety. CLEC-derived iPSCs were hypothesized to exhibit immune privileged properties similar to that of CLECs. See, for example, R. Saleh and H. M. Reza, ‘Short review on human umbilical cord lining epithelial cells and their potential clinical applications’, Stem Cell Res. Ther., vol. 8, no. 1, p. 222, October 2017, and R. H. G. Lim, J. X. K. Liew, A. Wee, J. Masilamani, S. K. Y. Chang, and T. T. Phan, ‘Safety Evaluation of Human Cord-Lining Epithelial Stem Cells Transplantation for Liver Regeneration in a Porcine Model’, Cell Transplant., vol. 29, p. 963689719896559, 2020, each of which is incorporated by reference in its entirety. (2) BJ-iPSCs were derived from BJ-fibroblasts with the use of modified mRNA. See, for example, Winanto, Z. J. Khong, B.-S. Soh, Y. Fan, and S.-Y. Ng, ‘Organoid cultures of MELAS neural cells reveal hyperactive Notch signaling that impacts neurodevelopment’, Cell Death Dis., vol. 11, no. 3, Art. no. 3, March 2020, which is incorporated by reference in its entirety. Tissue culture plates were coated with 3 mL of Matrigel for a minimum of 15 min at 37 °C before passaging. Briefly, the cells had a daily change of medium and were passaged via ReleSR™ (STEMCELL Technologies, Canada) upon reaching 70-80% confluency.

### SCPC Differentiation

Spinal Cord Progenitor Cells (SCPC) were differentiated from the CLEC23-iPSC and BJ-iPSC cell lines. The method for generating SCPCs is derived from. See, for example, S. H. Tay, Winanto, Z. J. Khong, Y. H. Koh, and S. Y. Ng, ‘Generation of Cortical, Dopaminergic, Motor, and Sensory Neurons from Human Pluripotent Stem Cells’, in Methods in Molecular Biology. New York, NY: Springer US, 2021, which is incorporated by reference in its entirety. Briefly, iPSCs were lifted off tissue culture dishes at 70-80% confluency with Accutase (Nacalai Tesque Inc., Japan) and plated onto Matrigel-coated (1 mL of 83.3 μg/mL for a minimum of 15 min at 37 °C) tissue culture plastic 6-well plates at 800,000 cells/well with neural induction media (NIM) supplemented with ROCK inhibitor Y-27632 (ROCKi, 5 μM, Miltenyi Biotech). NIM consists of DMEM/F12 (50%, Thermo Fisher Scientific, Waltham, MA, USA), neural medium (50%, Miltenyi Biotech), NeuroBrew-21 (1x, Miltenyi Biotech), N2 (1x, Miltenyi Biotech), Non-Essential Amino Acid (1x, Thermo Fisher Scientific), Glutamax (0.5x, Thermo Fisher Scientific), LDN-193189 (0.5 μM, Miltenyi Biotech) and CHIR-99021 (CHIR, 4.25 μM, Miltenyi Biotech). On day 3, retinoic acid (RA, 10 μM, Sigma, USA) was added to the NIM to induce caudalisation. On day 4, the semi-differentiated iPSCs were lifted off with Accutase and transferred onto a Matrigel-coated tissue culture plastic dish at 2.5×10⁶ cells/dish with NIM+RA and ROCKi. On day 5, NIM+RA and ROCKi were aspirated and replenished with NIM+RA. From day 6 to day 9, the cells had a daily media change of NIM+RA. On day 10, the differentiated cells were lifted off the dish with Accutase and subsequently characterized with various assays.

### MRR Measurement

The apparatus was established in previous works done within the same group. See, for example, W. K. Peng, L. Chen, B. O. Boehm, J. Han, and T. P. Loh, ‘Molecular phenotyping of oxidative stress in diabetes mellitus with point-of-care NMR system’, Npj Aging Mech. Dis., vol. 6, no. 1, Art. no. 1, October 2020, W. K. Peng et al., ‘Micromagnetic resonance relaxometry for rapid label-free malaria diagnosis’, Nat. Med., vol. 20, no. 9, pp. 1069-1073, September 2014, and S. S. Thamarath et al., ‘Rapid and Live-Cell Detection of Senescence in Mesenchymal Stem Cells by Micro Magnetic Resonance Relaxometry’, Stem Cells Transl. Med., p. szad014, Marxh 2023, each of which is incorporated by reference in its entirety. Briefly, the MRR system consists of a portable, permanent magnet (Metrolab Instruments, Switzerland) with B₀=0.5 T and a bench-top type nuclear magnetic resonance (NMR) console (Kea Magritek, New Zealand). MRR measurements of ¹H were performed with a resonance frequency of 21.015 MHz inside the magnet. MRR samples are loaded into a microcapillary tube (Fisherbrand, USA) fitted into a 900-μm inner diameter detection micro-coil within the single resonance proton MRR probe. Electronic components and the coil are all parts of a printed circuit board in the MRR probe (FIG. 1). Every MRR experiment is performed at a temperature of 26.3 °C within the magnet, as maintained by a temperature controller (RS component, UK).

For all MRR experiments, the concentration of iPSCs and SCPCs used was fixed at 180,000 cells in 4 μL volume. iPSC or SCPC samples were spun down at 300 g for 5 min and 1000 rpm at 3 min, respectively, with the supernatant being aspirated subsequently. PBS was then added, and the tube was spun down at 1500 rpm for 3 min. The supernatant was aspirated, and the cell pellet was transferred to an Eppendorf tube. The number of cells was counted (INCYTO, South Korea), and a sample of 45,000 cells/mL in 50 mL of cell solution was prepared. 4 mL of cell solution was pipetted into a micro-capillary tube, sealed with critoseal (Leica Microsystems) and mounted into the MRR system to obtain a readout. The measurement of proton transverse relaxation times (T₂) was performed by a standard Carr-Purcell-Meiboom-Gill (CPMG) pulse program (FIG. 3A). For all experiments, an inter-echo time of 500 μs with 4000 echoes was applied in the CPMG train of pulses. To allow sufficient time for all spins to return to thermal equilibrium, a recycle delay of 3 s was applied. Signal averaging was performed with a total of 24 scans for all MRR experiments.

To quantify the changes in T₂ during this differentiation process, T₂ of iPSCs and SCPCs were measured by MRR on day 1, 4 and 10 of the differentiation process.

### Immunofluorescent Staining

SCPCs were seeded on Matrigel-coated 96-well plates at 80,000 cells/well. The cells were stained with SOX1, HOXB4 and OCT4 antibodies. Briefly, SCPCs were fixed with 4% Paraformaldehyde (PFA, Biotium, USA) for 15 min. The fixative was removed and washed twice with PBS. The fixed wells were then permeabilized with 0.1% Triton-X in PBS for 15 min. After permeabilization, blocking buffer (2% Bovine Serum Albumin (BSA), 5% Fetal Bovine Serum) was added and incubated at room temperature for 1 h. Thereafter, primary antibodies were added and the samples were incubated at 4 °C overnight. The primary antibodies used were: SOX1 (1:250, Cell Signalling Technology), HOXB4 (1:250, Abcam) and OCT4 (1:500, Santa Cruz Biotechnology). PBS was used to wash the wells twice after removal of primary antibodies. Secondary antibodies with the corresponding host species and DAPI were then added to the wells and incubated at room temperature without light exposure for 1 h. The secondary antibodies and DAPI used were: donkey AlexaFluor488-conjugated anti-rabbit IgGs (1:500, Thermo Fisher Scientific), donkey AlexaFluor555-conjugated anti-Mouse IgGs (1:500, Thermo Fisher Scientific) and DAPI (1:1000, Thermo Fisher Scientific). PBS was used to wash the wells twice after the removal of primary antibodies. The stained cells were imaged using a Leica DMi8 Microscope and quantified using CellProfiler.

### Flow Cytometry Analysis

SCPCs were collected into 15 mL centrifuge tubes and adjusted to a density of 5×10⁶ cells/mL. The cells were then fixed in 4% PFA for 15 min at room temperature. Once the cells were fixed, they were centrifuged at 3000 rpm for 5 min and washed once with PBS. Thereafter, the cells were stained by incubation at room temperature for a minimum of 2 h with primary antibodies reconstituted in permeabilization/blocking buffer (0.5% saponin, 1% BSA). The primary antibodies used were: SOX1 (1:250, Cell Signalling Technology), Nestin (1:200, Abcam), HOXB4 (1:250, Abcam) and OCT4 (1:500, Santa Cruz Biotechnology). After staining, the cells were centrifuged at 3000 rpm for 5 min and washed twice with PBS. They were then stained by secondary antibodies reconstituted in permeabilization/blocking buffer in the absence of light and at room temperature for 45 min. The secondary antibodies used were: donkey AlexaFluor488-conjugated anti-rabbit IgGs (1:500, Thermo Fisher Scientific) and donkey AlexaFluor555-conjugated anti-Mouse IgGs (1:500, Thermo Fisher Scientific). Lastly, the cells were centrifuged at 3000 rpm for 5 min, washed once with PBS and resuspended in 300 μL PBS. Cytoflex flow cytometer (Beckman Coulter, USA) was used to analyze the cells.

iPSC Spiking and Colony Culture Assay

SCPCs that were harvested were spiked with iPSCs at different percentages, including 1%, 5% and 10%. Thereafter, the SCPCs were counted, and a fixed number of cells was removed to account for the addition of iPSCs. The cells were seeded in a 6-well plate at an initial density of 1×10⁷ cells/well. They were cultured in 2 mL of hiPSC medium for six days with a daily change of the medium. Cells were fixed and stained with DAPI and OCT4 antibodies before imaging with a Leica DMi8 microscope. Colony sizes were analyzed with an ImageJ particle size analyzer.

Fe³⁺ Staining and Quantification of iPSCs and SCPCs

Quantification of intracellular iron (Fe³⁺) in iPSCs and SCPCs was performed via a reversible fluorescent Fe³⁺ sensor (RPE). See, for example, Y. Wei, Z. Aydin, Y. Zhang, Z. Liu, and M. Guo, ‘A Turn-on Fluorescent Sensor for Imaging Labile Fe3+ in Live Neuronal Cells at Subcellular Resolution’, ChemBioChem, vol. 13, no. 11, pp. 1569-1573, July 2012, which is incorporated by reference in its entirety. For Fe³⁺ staining, a stock solution of RPE (1 mM in acetonitrile) was diluted to a concentration of 20 μM in PBS. 3×10⁵ suspended cells were harvested and centrifuged to remove the supernatant. The cells were then incubated with PBS containing RPE (20 μM) at 37 °C for 20 min. After incubation, the cells were washed twice and suspended in PBS for measurement of their fluorescent intensity via flow cytometry.

### Statistical Analysis

Statistical analyses were performed using GraphPad Prism 9. All data were presented as the mean±standard deviation (SD). p value<0.05 was considered statistically significant, *: p<0.05; **: p<0.01; ***: p<0.001; ****: p<0.0001.

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

1. A method of evaluating induced pluripotent stem cells and cells differentiated therefrom, comprising:
loading a sample including a plurality of induced pluripotent stem cells and cells differentiated therefrom in a sensor;
placing the sensor including the sample within or nearby a detection coil of a magnetic resonance relaxometry device;
determining a T2 value for the sample; and
evaluating a quality of the induced pluripotent stem cells and cells differentiated therefrom, from the T2 value.

2. The method according to claim 1, wherein evaluating includes comparing the T2 value of the induced pluripotent stem cells and cells differentiated therefrom to a T2 value of induced pluripotent stem cells measured at an earlier time.

3. The method according to claim 1, further comprising passaging induced pluripotent stem cells and cells differentiated therefrom, measuring a T2 value of the passaged induced pluripotent stem cells and cells differentiated therefrom to assess variation between the passaged induced pluripotent stem cells and cells differentiated therefrom from the induced pluripotent stem cells and cells differentiated therefrom.

4. The method according to claim 1, wherein the sample is a pellet including induced pluripotent stem cells and cells differentiated therefrom in the sensor.

5. The method according to claim 1, wherein the sample includes a cluster of induced pluripotent stem cells and cells differentiated therefrom.

6. The method according to claim 5, wherein the cluster has a size of at least 50 microns.

7. The method according to claim 1, wherein the T2 value correlates to a doubling time for the induced pluripotent stem cells.

8. The method according to claim 1, wherein the T2 value correlates to the percentage of differentiated progenitor cells.

9. The method according to claim 1, wherein the T2 value correlates to a percentage of differentiated progenitor cells.

10. The method according to claim 1, wherein the T2 value correlates to a percentage of undifferentiated, residual iPSCs in a differentiated cell population.

11. The method according to claim 1, wherein the T2 value correlates to an ability of iPSCs to differentiate into progenitor cells or fully differentiated cells.

12. The method according to claim 1, wherein the T2 value correlates to OCT4 expression.

13. The method according to claim 1, wherein the T2 value correlates to an EdU colony forming assay.

14. The method according to claim 1, wherein evaluating a quality of the induced pluripotent stem cells and cells differentiated therefrom, from the T2 value occurs on day 1 of a differentiation process.

15. A system for evaluating induced pluripotent stem cells and cells differentiated therefrom comprising:
a magnetic resonance relaxometry device configured to determining a T2 value for a sample including a plurality of induced pluripotent stem cells and cells differentiated therefrom in a sensor and evaluating induced pluripotent stem cells and cells differentiated therefrom by monitoring the T2 value of the sample.

16. The system according to claim 15, further comprising a cell passaging device as a source for the plurality of induced pluripotent stem cells and cells differentiated therefrom.

17. The system according to claim 15, wherein evaluating includes comparing the T2 value of the induced pluripotent stem cells and cells differentiated therefrom to a T2 value of induced pluripotent stem cells and cells differentiated therefrom measured at an earlier time.

18. The system according to claim 15, wherein a T2 value of passaged induced pluripotent stem cells and cells differentiated therefrom is measured to assess variation between the passaged induced pluripotent stem cells from the induced pluripotent stem cells and cells differentiated therefrom.

19. The system according to claim 15, wherein the sample is a pellet including induced pluripotent stem cells and cells differentiated therefrom in the sensor.

20-29. (canceled)
